SEQUENCE LISTING (2 sequences)

SEQ ID NO: 1 - DNA, 1152 bp, ARTIFICIAL SEQUENCE
  SYNTHETIC PRIMER-CTLA4Ig
atgggtgtactgctcacacagaggacgctgctcagtctggtccttgcactcctgtttccaagcatggcgagcatggcaatgcacgtggcccagcctgctgtggtactggccagcagccgaggcatcgctagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgatcaggagcccaaatcttctgacaaaactcacacatccccaccgtccccagcacctgaactcctgggtggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 383 aa, ARTIFICIAL SEQUENCE
  SYNTHETIC PEPTIDE-CTLA4Ig
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2017/0042972 A1
(43) Pub. Date: Feb. 16, 2017

(54) USE OF CTLA4 COMPOUND FOR ACHIEVING DRUG-FREE REMISSION IN SUBJECTS WITH EARLY RA

(71) Applicant: Chetan KARYEKAR, Princeton, NJ (US)

(72) Inventor: Chetan KARYEKAR, Princeton, NJ (US)

(21) Appl. No.: 15/306,198

(22) PCT Filed: Apr. 23, 2015
(86) PCT No.: PCT/US15/27281

§ 371 Date: Oct. 24, 2016

Related U.S. Application Data
(60) Provisional application No. 61/984,287, filed on Apr. 25, 2014.

(51) Int. Cl. A61K 38/17; A61K 9/00; G01N 33/564; C07K 14/705

(52) U.S. Cl. CPC A61K 38/1774; C07K 14/70503; A61K 9/0019; G01N 33/564; C07K 2319/30; G01N 2800/102

(57) ABSTRACT

The present invention is directed to methods and compositions for achieving drug-free remission in subjects with early RA by administering to a subject in need thereof an effective amount of soluble CTLA4 molecule until Disease Activity Score Calculator for Rheumatoid Arthritis (DAS)-defined remission is achieved and then withdrawing the RA therapy.

### CROSS-REFERENCE TO RELATED APPLICATIONS

This application claims priority to U.S. Provisional Application Ser. No. 61/984,287, filed Apr. 25, 2014; the entire content of which is incorporated herein by reference.

### FIELD OF THE INVENTION

The present invention relates generally to the field of rheumatoid arthritis (RA), e.g., early rheumatoid arthritis. In particular, the invention relates to methods and compositions for achieving drug-free remission in subjects with early RA by administering to a subject in need thereof an effective amount of soluble CTLA4 molecule until Disease Activity Score Calculator for Rheumatoid Arthritis (DAS)-defined remission is achieved and then withdrawing the RA therapy.

### BACKGROUND OF THE INVENTION

Rheumatoid arthritis (RA) is the most common inflammatory arthritis, affecting approximately 1% of the population worldwide (Wolfe, F., “The epidemiology of drug treatment failure in rheumatoid arthritis”, Baillieres Clin. Rheumatol., 9(4):619-632 (November 1995)). Women are 2-3 times more likely to develop disease compared to men, with a peak incidence between the fourth and sixth decades of life (Hochberg, M. C. et al., “Epidemiology of rheumatoid arthritis: update”, Epidemiol. Rev., 12:247-252 (1990); Markenson, J. A., “Worldwide trends in the socioeconomic impact and long-term prognosis of rheumatoid arthritis”, Semin. Arthritis Rheum., 21(2 Suppl. 1):4-12 (October 1991); Spector, T. D., “Rheumatoid arthritis”, Rheum. Dis. Clin. North Am., 16(3):513-537 (August 1990); and Zvaifler, N. J., “Etiology and pathogenesis of rheumatoid arthritis”, in Arthritis and Allied Conditions, pp. 723-736, McCarty, D. J. et al., eds., Lea & Febiger, Philadelphia (1993)). While RA is recognized clinically because of the severe inflammation affecting the synovial joints, it is also a systemic disease with frequent extra-articular manifestations. The natural history of RA is unfortunately characterized by joint destruction, impaired physical function and poor health related quality of life.

There is increasing scientific evidence that joint destruction occurs early in RA. Over 90% of subjects have evidence of joint damage by conventional radiography within two years after the diagnosis of RA (Emery, P., “The Optimal Management of Early Rheumatoid Disease: The Key to Preventing Disability”, Br. J. Rheum., 33:765-768 (1994)). Joint damage can be detected within weeks of the onset of symptoms using more sensitive techniques such as MRI or ultrasound (McGonagle, D. et al., “The relationship between synovitis and bone changes in early untreated rheumatoid arthritis”, Arthritis Rheum., 42:1706-1711 (1999) and Wakefield, R. J. et al., “The value of sonography in the detection of bone erosion in patients with rheumatoid arthritis: A comparative study with conventional radiography”, Arthritis Rheum., 43:2761-2770 (2000)). These findings have created an increasing need for therapies which can effectively inhibit the inflammatory processes which cause bone and cartilage loss early on in RA and have placed increasing emphasis on earlier diagnosis and treatment of RA.

The normal synovium is a tissue that surrounds and separates joint spaces. The lining layer of cells, composed of macrophage-like and fibroblast-like synoviocytes, overlays a thin connective tissue stroma containing sparse numbers of dendritic cells, fibroblasts, mast cells and vascular structures (Konttinen, Y. T. et al., “Characterization of the immunocompetent cells of rheumatoid synovium from tissue sections and eluates”, Arthritis Rheum., 24(1):71-79 (January 1981)).

In RA, the synovial tissue becomes markedly thickened and swollen. As the disease progresses, there is gradual proliferation and recruitment of synoviocytes, as well as recruitment of inflammatory cells into the synovium (Konttinen, Y. T. et al., “Characterization of the immunocompetent cells of rheumatoid synovium from tissue sections and eluates”, Arthritis Rheum., 24(1):71-79 (January 1981)). Up to 50% of the infiltrating leukocytes in the synovium are T-lymphocytes, primarily CD4+ T cells with an activated/memory phenotype (Konttinen, Y. T. et al., “Characterization of the immunocompetent cells of rheumatoid synovium from tissue sections and eluates”, Arthritis Rheum., 24(1):71-79 (January 1981); Forre, O. et al., “Augmented numbers of HLA-DR-positive T lymphocytes in the synovial fluid and synovial tissue of subjects with rheumatoid arthritis and juvenile rheumatoid arthritis: in vivo-activated T lymphocytes are potent stimulators in the mixed lymphocyte reaction”, Scand. J. Immunol., 15(2):227-231 (February 1982); Van-Boxel, J. A. et al., “Predominantly T-cell infiltrate in rheumatoid synovial membranes”, N. Engl. J. Med., 293(11):517-520 (September 1975); Kidd, B. L. et al., “Immunohistological features of synovitis in ankylosing spondylitis: a comparison with rheumatoid arthritis”, Ann. Rheum. Dis., 48(2):92-98 (February 1989); Cush, J. J. et al., “Phenotypic analysis of synovial tissue and peripheral blood lymphocytes isolated from subjects with rheumatoid arthritis”, Arthritis Rheum., 31(10):230-238 (October 1988); Laffon, A. et al., “Upregulated expression and function of VLA-4 fibronectin receptors on human activated T cells in rheumatoid arthritis”, J. Clin. Invest., 88(2):546-552 (August 1991); and Klareskog, L. et al., “Relationship between HLA DR expressing cells and T lymphocytes of different subsets in rheumatoid synovial tissue”, Scand. J. Immunol., 15(5):501-507 (May 1981)). Cells of monocyte/macrophage origin also become prominent in the rheumatoid synovium, accounting for up to 20% of cells, and they too exhibit an activated phenotype (Firestein, G. S. et al., “How important are T cells in chronic rheumatoid synovitis?”, Arthritis Rheum., 33(6):768-773 (June 1990) and Firestein, G. S. et al., “Quantitative analysis of cytokine gene expression in rheumatoid”, J. Immunol., 144(9):3347-3353 (May 1, 1990)). Monocyte/macrophage-like cells in the rheumatoid synovium produce an array of pro-inflammatory molecules, including the cytokines IL-1, TNF-α, IL-6, GM-CSF as well as proteolytic enzymes including collagenases and matrix metalloproteinases. B-cells, plasma cells and neutrophils account for less than 5% of cells in the rheumatoid synovium, although neutrophils are prominent in the synovial fluid (Konttinen, Y. T. et al., “Characterization of the immunocompetent cells of rheumatoid synovium from tissue sections and eluates”, Arthritis Rheum., 24(1):71-79 (January 1981); Forre, O. et al., “Augmented numbers of HLA-DR-positive T lymphocytes in the synovial fluid and synovial tissue of subjects with rheumatoid arthritis and juvenile rheumatoid arthritis: in vivo-activated T lymphocytes are potent stimulators in the mixed lymphocyte reaction”, Scand. J. Immunol., 15(2):227-231 (February 1982); and Firestein, G. S. et al., “Quantitative analysis of cytokine gene expression in rheumatoid”, J. Immunol., 144(9):3347-3353 (May 1, 1990)).

As synovial proliferation and inflammation advances, the expanding mass of vascular, inflammatory synovial tissue is termed pannus. Pannus is responsible for invading articular cartilage and destroying bone. The products of activated T cells are felt to be the driving factors behind the formation and expansion of pannus (Zvaifler, N. J. et al., “Alternative models of joint destruction in rheumatoid arthritis”, Arthritis Rheum., 37(6):783-789 (June 1994)).

The monocyte/macrophage-like cells and dendritic cells in the rheumatoid synovium express both class II MHC as well as costimulatory molecules such as CD80 (B7-1)/CD86 (B7-2), and presumably function as antigen presenting cells (Balsa, A. et al., “Differential expression of the costimulatory molecules B7.1 (CD80) and B7.2 (CD86) in rheumatoid synovial tissue”, Br. J. Rheumatol., 35(1):33-37 (January 1996); Liu, M. F. et al., “The presence of costimulatory molecules CD86 and CD28 in rheumatoid arthritis synovium”, Arthritis Rheum., 39(1):110-114 (January 1996); Ranheim, E. A. et al., “Elevated expression of CD80 (B7/BB1) and other accessory molecules on synovial fluid mononuclear cell subsets in rheumatoid arthritis”, Arthritis Rheum., 37(11):1637-1646 (November 1994); Sflkakis, P. P. et al., “Expression of CD28, CTLA4, CD80, and CD86 molecules in subjects with autoimmune rheumatic diseases: implications for immunotherapy”, Clin. Immunol. Immunopathol., 83(3):195-198 (June 1997); and Thomas, R. et al., “Functional differentiation of dendritic cells in rheumatoid arthritis: role of CD86 in the synovium”, J. Immunol., 156(8):3074-3086 (Apr. 15, 1996)). Activated CD4+ T cells expressing CD28 are prominent infiltrating cell types in the rheumatoid synovium and commonly are found adjacent to cells that express class II MHC and costimulatory molecules. This suggests an important role for T cell activation/costimulation in the pathogenesis of synovial inflammation. This is consistent with the experimental observation that activated T cells, either through cell to cell contact with synoviocytes and osteoclasts or by the elaboration of secreted cytokines, are important factors in driving synovitis and bone destruction in RA. Taken together, these observations suggest that activated T cells and the costimulatory signals delivered through CD28 play a key role in driving the immunopathology of RA.

There may be a “window of opportunity” in early RA to alter the course of disease if tightly controlled, which diminishes once the inflammatory processes are more established (Cush, J. J., “Early rheumatoid arthritis—is there a window of opportunity?”, J. Rheumatol. (Suppl.), 80:1-7 (2007). This could aid decisions on the use of a combination of biologic disease-modifying anti-rheumatic drugs (DMARDs) and conventional synthetic (cs)DMARDs versus step-up therapy in early RA (Singh, J. A. et al., “2012 update of the 2008 American College of Rheumatology recommendations for the use of disease-modifying antirheumatic drugs and biologic agents in the treatment of rheumatoid arthritis”, Arthritis Care Res. (Hoboken), 64:625-639 (2012)). Once RA is well controlled, the ability to sustain remission following the withdrawal of immunomodulatory medications would be an indication of disease modification.

Remission following withdrawal or tapering of RA therapy is an important goal in early RA. Previous studies have examined a variety of treatment withdrawal paradigms with a number of biologic agents but have not demonstrated sustained remission following the rapid withdrawal of all RA treatment(Huizinga, T. et al., “Clinical and radiographic outcomes at two years and the effect of tocilizumab discontinuation following sustained remission in the second year of the ACT-RAY study”, Ann. Rheum. Dis., 72(Suppl 3):63 (2013); Quinn, M. A. et al., “Very early treatment with infliximab in addition to methotrexate in early, poor-prognosis rheumatoid arthritis reduces magnetic resonance imaging evidence of synovitis and damage, with sustained benefit after infliximab withdrawal”, Arthritis Rheum., 52:27-35 (2005); van den Broek, M. et al., “Discontinuation of infliximab and potential predictors of persistent low disease activity in patients with early rheumatoid arthritis and disease activity score-steered therapy”, Ann. Rheum. Dis., 70:1389-1394 (2011); Villeneuve, E. et al., “Preliminary results of a multicentre randomized controlled trial of etanercept and methotrexate to induce remission in patients with newly diagnosed inflammatory arthritis”, Arthritis Rheum., 63:S960-S961 (2011)). Most anti-tumor necrosis factor withdrawal studies maintained MTX or maintained the biologic at half dose. (Detert, J. et al., “Induction therapy with adalimumab plus methotrexate for 24 weeks followed by methotrexate monotherapy up to week 48 versus methotrexate therapy alone for DMARD-naive patients with early rheumatoid arthritis”, Ann. Rheum. Dis., 72:844-850 (2013); Emery, P. et al., “Assessing maintenance of remission after withdrawal of etanercept plus methotrexate, methotrexate alone, or placebo in early rheumatoid arthritis patients who achieved remission with etanercept and methotrexate”, Arthritis Rheum., 65(Suppl 10):2689 (2013); Nam, J. L. et al., “Remission induction comparing infliximab and high-dose intravenous steroid, followed by treat-to-target: a double-blind, randomized, controlled trial in new-onset, treatment-naive, rheumatoid arthritis”, Ann. Rheum. Dis., 73:75-85 (2014); Nishimoto, N. et al., “Drug free REmission/low disease activity after cessation of tocilizumab (Actemra) Monotherapy (DREAM) study”, Mod. Rheumatol., 24:17-25 (2014); Smolen, J. S. et al., “Maintenance, reduction, or withdrawal of etanercept after treatment with etanercept and methotrexate in patients with moderate rheumatoid arthritis”, Lancet, 381:918-929 (2013); Smolen, J. S. et al., “Adjustment of therapy in rheumatoid arthritis on the basis of achievement of stable low disease activity with adalimumab plus methotrexate or methotrexate alone”, Lancet, 383:321-332 (2014)).

The approach of withdrawing all therapy and maintaining complete drug-free remission is an ideal treatment benefit for patients. Additionally, physicians can justify the economic burden of treating patients with early RA, especially if patients who are likely to achieve drug-free remission can be identified prospectively.

### SUMMARY OF THE INVENTION

The present invention provides a method of achieving drug-free remission in subjects with early RA comprising administering to the subject in need thereof an effective amount of the CTLA4 molecule or pharmaceutical composition thereof, achieving DAS-defined remission and then withdrawing the RA therapy.

The present invention also provides a method of achieving drug-free remission in subjects with early RA comprising administering to the subject in need thereof an effective amount of the CTLA4Ig molecule or pharmaceutical composition thereof, achieving DAS-defined remission and then withdrawing the RA therapy.

In the method of the present invention DAS-defined remission is characterized as DAS28 (C-reactive protein[CRP]) less than 2.6 after 12 months of treatment.

In the method of the present invention the CTLA4 pharmaceutical composition is a subcutaneous formulation, which is administered at 125 mg/week subcutaneously.

The present invention also provides a method of identifying subjects likely to achieve sustained drug-free remission. The subjects with early RA who are likely to achieve sustained drug-free remission are characterized as having active clinical synovitis of ≧2 joints for ≧8 weeks, DAS28 (CRP) ≧3.2 and anti-citrullinated peptide (CCP)-2 antibody positivity.

The methods of the present invention also may be used to inhibit structural damage in subjects with early RA in drug-free remission as assessed by erosion, osteitis and/or synovitis scoring of the wrist and hand.

### BRIEF DESCRIPTION OF THE DRAWINGS

FIG. 1 presents the nucleotide sequence (SEQ ID NO:1) of a portion of an expression cassette for a CTLA4-Ig molecule. Also shown is the amino acid sequence (SEQ ID NO:2) encoded by the nucleic acid. CTLA4-Ig molecules that can be produced from this expression cassette include molecules having the amino acid sequence of residues: (i) 26-383 of SEQ ID NO:2, (ii) 26-382 of SEQ ID NO:2, (iii) 27-383 of SEQ ID NO:2, or (iv) 26-382 of SEQ ID NO:2, or optionally (v) 25-382 of SEQ ID NO:2, or (vi) 25-383 of SEQ ID NO:2. The expression cassette comprises the following regions: (a) an Oncostatin M signal sequence (nucleotides 11-88 of SEQ ID NO: 1; amino acids 1-26 of SEQ ID NO:2); (b) an extracellular domain of human CTLA4 (nucleotides 89-463 of SEQ ID NO:1; amino acids 27-151 of SEQ ID NO:2); (c) a modified portion of the human IgG1 constant region (nucleotides 464-1159 of SEQ ID NO: 1; amino acids 152-383 of SEQ ID NO:2), including a modified hinge region (nucleotides 464-508 of SEQ ID NO:1; amino acids 152-166 of SEQ ID NO:2), a modified human IgG1 CH2 domain (nucleotides 509-838 of SEQ ID NO:1; amino acids 167-276 of SEQ ID NO:2), and a human IgG1 CH3 domain (nucleotides 839-1159 of SEQ ID NO:1; amino acids 277-383 of SEQ ID NO:2).

FIG. 2 shows the study design of the AVERT clinical study described in Example III. CRP=C-reactive protein; DAS=Disease Activity Score; MRI=magnetic resonance imaging; MTX=methotrexate; RA=rheumatoid arthritis.

FIG. 3 shows the Patient Disposition Flow Chart utilized in the AVERT clinical study described in Example III. MTX=methotrexate.

FIG. 4A-D shows efficacy outcomes over time in the AVERT clinical study described in Example III. 4A shows the proportion of patients with Disease Activity Score (DAS)-defined remission (DAS28 [C-reactive protein, CRP]<2.6). 4B shows the proportion of patients with Simplified Disease Activity Index remission (≦3.3). 4C shows the proportion of patients with Boolean remission (tender joint count ≦1, swollen joint count ≦1, patient global assessment of disease activity ≦1 [0-10 scale], high-sensitivity CRP ≦1 mg/dL). 4D shows the major clinical response (ACR 70 response for a minimum of 6 consecutive months at any time period prior to the time point). Error bars represent 95% confidence intervals. Missing remission data not due to premature discontinuation and not at Day 1 of the treatment period or at Day 169 of the withdrawal period was imputed as a remission if the missing value occurred between two observed remissions. Missing ACR response data not due to premature discontinuation and not at Day 1 of the treatment period or at Day 169 of the withdrawal period was imputed as an ACR response if the missing value occurred between two observed ACR responses. ACR=American College of Rheumatology, CRP=C-reactive protein, DAS=Disease Activity Score, MTX=methotrexate, SDAI=Simplified Disease Activity Index.

FIG. 5A-E shows the additional efficacy outcomes over time in the AVERT clinical study described in Example III. 5A shows the proportion of patients with Clinical Disease Activity index remission (≦2.8). 5B shows the proportion of patients with American college of Rheumatology (ACR) 20. 5C shows the proportion of patients with ACR 50. 5D shows the proportion of patients with ACR 70. 5E shows the proportion of patients with ACR 90. Error bars represent 95% confidence intervals. Missing remission data not due to premature discontinuation and not at Day 1 of the treatment period or at Day 169 of the withdrawal period was imputed as a remission if the missing value occurred between two observed remissions. Missing ACR response data not due to premature discontinuation and not at Day 1 of the treatment period or at Day 169 of the withdrawal period was imputed as an ACR response if the missing value occurred between two observed ACR responses. ACR=American College of Rheumatology; CDAI=Clinical Disease Activity Index; MTX=methotrexate.

FIG. 6 shows the proportion of patients in Disease Activity Score (DAS)-defined remission (DAS28 [C-reactive protein; CRP]<2.6) during the withdrawal period of AVERT clinical study described in Example III. Missing remission data not due to premature discontinuation and not at Day 1 of the treatment period or at Day 169 of the withdrawal period was imputed as a remission if the missing value occurred between two observed remissions.

FIG. 7A-C shows the progression through magnetic resonance imaging of subjects in the AVERT clinical study described in Example III. 7A shows the adjusted mean change from baseline in total synovitis score. 7B shows the adjusted mean change from baseline in total osteitis score. 7C shows the adjusted mean change from baseline in total erosion score. Error bars represent standard error. MTX=methotrexate.

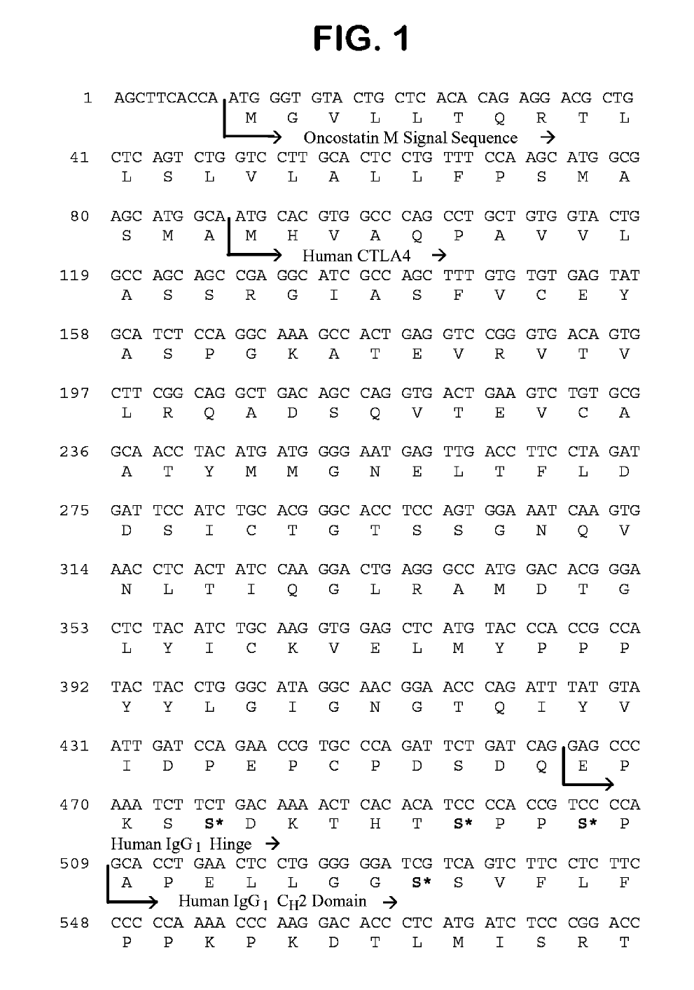

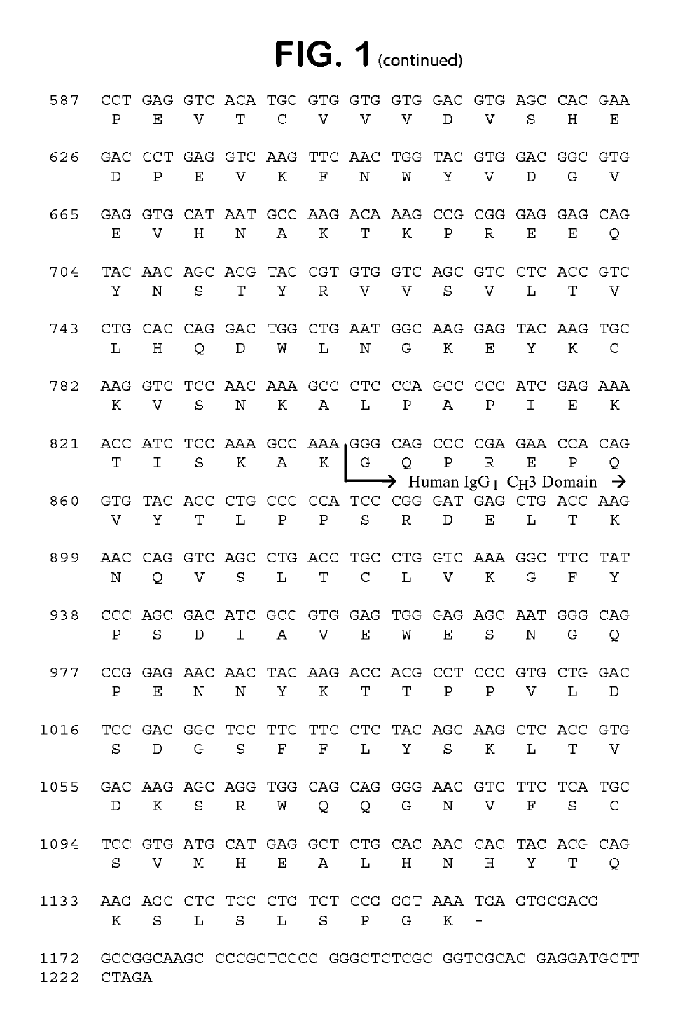

### DETAILED DESCRIPTION OF THE INVENTION

As utilized herein:

The terms “CTLA4-Ig molecule” or “CTLA4Ig molecule” refer to a protein molecule that comprises at least a polypeptide having a CTLA4 extracellular domain or portion thereof and an immunoglobulin constant region or portion thereof. The extracellular domain and the immunoglobulin constant region can be wild-type, or mutant or modified, and mammalian, including human or mouse. The polypeptide can further comprise additional protein domains. A CTLA4-Ig molecule can also refer to multimer forms of the polypeptide, such as dimers, tetramers, and hexamers. A CTLA4-Ig molecule also is capable of binding to CD80 and/or CD86.

The term “B7-1” refers to CD80; the term “B7-2” refers CD86; and the term “B7” refers to both B7-1 and B7-2 (CD80 and CD86). The term “B7-1-Ig” or “B7-1Ig” refers to CD80-Ig; the term “B7-2-Ig” or “B7-2Ig” refers CD86-Ig.

In one embodiment, “CTLA4Ig” or “abatacept” refers to a protein molecule having the amino acid sequence of residues: (i) 26-383 of SEQ ID NO:2, (ii) 26-382 of SEQ ID NO:2; (iii) 27-383 of SEQ ID NO:2, or (iv) 27-382 of SEQ ID NO:2, or optionally (v) 25-382 of SEQ ID NO:2, or (vi) 25-383 of SEQ ID NO:2. In monomeric form these proteins can be referred to herein as “SEQ ID NO:2 monomers”, or monomers “having a SEQ ID NO:2 sequence”. These SEQ ID NO:2 monomers can dimerize, such that dimer combinations can include, for example: (i) and (i); (i) and (ii); (i) and (iii); (i) and (iv); (i) and (v); (i) and (vi); (ii) and (ii); (ii) and (iii); (ii) and (iv); (ii) and (v); (ii) and (vi); (iii) and (iii); (iii) and (iv); (iii) and (v); (iii) and (vi); (iv) and (iv); (iv) and (v); (iv) and (vi); (v) and (v); (v) and (vi); and, (vi) and (vi). These different dimer combinations can also associate with each other to form tetramer CTLA4Ig molecules. These monomers, dimers, tetramers and other multimers can be referred to herein as “SEQ ID NO:2 proteins” or proteins “having a SEQ ID NO:2 sequence”. (DNA encoding CTLA4Ig as shown in SEQ ID NO:2 was deposited on May 31, 1991 with the American Type Culture Collection (ATCC), 10801 University Blvd., Manassas, Va. 20110-2209 under the provisions of the Budapest Treaty, and has been accorded ATCC accession number ATCC 68629; a Chinese Hamster Ovary (CHO) cell line expressing CTLA4Ig as shown in SEQ ID NO:2 was deposited on May 31, 1991 with ATCC identification number CRL-10762).

A “drug substance” refers to the starting material utilized in formulation of the final drug product. Typical CTLA4Ig drug substance composition comprises a protein concentration from 20 mg/ml to 60 mg/ml, pH from 6 to 8 and % HMW species of <5%.

A “formulated bulk solution” refers to the final formulation prior to filling of the container such as the formulated solution prior to filling the vials for lyophilization, or the formulated solution prior to filling the syringe for SC injection.

A “drug product” refers to the final formulation packaged in a container which may be reconstituted before use, such as with a lyophilized drug product; diluted further before use, such as with a liquid drug product; or utilized as is, such as with a SC solution drug product.

“Health Questionnaire Assessments (HAQs)” refers to a set of questions used to evaluate patients for symptoms of disease activity. These symptoms included: joint swelling, joint tenderness, inflammation, morning stiffness, disease activity and disability evaluated by each patient in a self-administered questionnaire regarding their physical well-being and function, disease activity and disability as evaluated a physician, and pain (Fries, J. F. et al., J. Rheumatol., 9:789-793 (1982)).

“Medical Outcomes Study Short Form-36 (SF-36)” refers to forms used to evaluate the impact of therapy on health-related quality of life (HRQOL). The SF-36 consists of 36 items which covers four physical and four mental domains (physical function, role-physical, bodily pain, general health, vitality, social function, role emotional, and mental health). These individual domains are used to derive the physical and mental component summary scores which range from 0 to 100, with higher scores indicating better quality of life.

The term “ACR” refers to clinical response studies based on criteria established by the American College of Rheumatology. The ACR Core Data Set and Response Definitions are described in Table 1 below. A subject satisfies the “ACR20” criterion if there was a 20 percent improvement in tender and swollen joint counts and 20 percent improvement in three of five remaining symptoms measured, such as patient and physician global disease changes, pain, physical disability, and an acute phase reactant such as C-reactive Protein (CRP) or Expedited Safety Report (ESR) (Felson, D. T. et al., Arthritis Rheum., 36:729-740 (1993); Felson, D. T. et al., Arthritis Rheum., 38:1-9 (1995)). Similarly, a subject satisfies the “ACR50” or “ACR70” criterion if there was a 50 or 70 percent improvement, respectively, in tender and swollen joint counts and 50 or 70 percent improvement, respectively, in three of five remaining symptoms measured, such as patient and physician global disease changes, pain, physical disability, and an acute phase reactant such as CRP or ESR.

TABLE 1
ACR Core Data Set and Response Definitions

| ACR core data set component | Validated Measurement Tool |
|---|---|
| 1. Tender joint count | Standardized 68 joint count |
| 2. Swollen joint count | Standardized 66 joint count |
| 3. Subject global assessment of pain | A 0-100 mm visual analog scale |
| 4. Subject global assessment of | A 0-100 mm visual analog scale |
| disease activity | |
| 5. Physician global assessment of | A 0-100 mm visual analog scale |
| disease activity | |
| 6. Subject assessment of physical | Health Assessment Questionnaire |
| function | (HAQ) |
| 7. Acute phase reactant value | ESR (Westergren) and C-reactive |
| | protein |

Serum samples can be analyzed for CTLA4Ig by an enzyme-linked immunosorbent assay (ELISA).

### CTLA4-Ig Monomers and Multimers

CTLA4-Ig molecules can include, for example, CTLA4-Ig proteins in monomer, dimer, trimer, tetramer, pentamer, hexamer, or other multimeric forms. CTLA4-Ig molecules can comprise a protein fusion with at least an extracellular domain of CTLA4 and an immunoglobulin constant region. CTLA4-Ig molecules can have wild-type or mutant sequences, for example, with respect to the CTLA4 extracellular domain and immunoglobulin constant region sequences. CTLA4-Ig monomers, alone, or in dimer, tetramer or other multimer form, can be glycosylated.

In some embodiments, the invention provides populations of CTLA4-Ig molecules that have at least a certain percentage of dimer or other multimer molecules. For example, the invention provides CTLA4-Ig molecule populations that are greater than 90%, 95%, 96%, 97%, 98%, 99%, or 99.5% CTLA4-Ig dimers. In one embodiment, the invention provides a CTLA4-Ig molecule population that comprises from about 95% to about 99.5% CTLA4-Ig dimer and from about 0.5% to about 5% of CTLA4-Ig tetramer. In another embodiment, the CTLA4-Ig molecule population comprises about 98% CTLA4-Ig dimer, about 1.5% CTLA4-Ig tetramer and about 0.5% CTLA4-Ig monomer.

In one embodiment, the invention provides a population of CTLA4-Ig molecules wherein the population is substantially free of CTLA4-Ig monomer molecules. Substantially free of CTLA4-Ig monomer molecules can refer to a population of CTLA4-Ig molecules that have less than 1%, 0.5%, or 0.1% of monomers.

In one embodiment, the invention provides a population of CTLA4-Ig molecules wherein the population is substantially free of CTLA4-Ig multimers that are larger than dimers, such as tetramers, hexamers, etc. Substantially free of CTLA4-Ig multimer molecules larger than dimers can refer to a population of CTLA4-Ig molecules that have less than 6%, 5%, 4%, 3%, 2%, 1%, 0.5%, or 0.1% of CTLA4-Ig multimers larger than dimeric form.

In one embodiment, a CTLA4-Ig monomer molecule can have, for example, the amino acid sequence of: (i) 26-383 of SEQ ID NO:2, (ii) 26-382 of SEQ ID NO:2 (iii) 27-383 of SEQ ID NO:2, or (iv) 27-382 of SEQ ID NO:2, or optionally (v) 25-382 of SEQ ID NO:2, or (vi) 25-383 of SEQ ID NO:2. When an expression cassette comprising the nucleic acid sequence of SEQ ID NO: 1 is expressed in CHO cells, the predominant monomer form expressed has the N-terminus amino acid residue of methionine (residue 27 of SEQ ID NO:2), which corresponds to the N-terminus amino acid residue of wild-type human CTLA4. However, because SEQ ID NO:1 also includes the coding sequence for an Oncostatin M Signal Sequence (nucleotides 11-88 of SEQ ID NO: 1), the expressed protein from SEQ ID NO:1 contains an Oncostatin M Signal Sequence. The signal sequence is cleaved from the expressed protein during the process of protein export from the cytoplasm, or secretion out of the cell. But cleavage can result in N-terminal variants, such as cleavage between amino acid residues 25 and 26 (resulting in an N-terminus of residue 26, i.e., the “Ala variant”), or between amino acid residues 24 and 25 (resulting in an N-terminus of residue 2, i.e., the “Met-Ala variant”), as opposed to cleavage between amino acid residues 26 and 27 (resulting in an N-terminus of residue 27). For example, the Met-Ala variant can be present in a mixture of CTLA4-Ig molecules at about 1%, and the Ala variant can be present in a mixture of CTLA4-Ig molecules at about 8-10%. In addition, the expressed protein from SEQ ID NO:1 can have C-terminus variants due to incomplete processing. The predominant C-terminus is the glycine at residue 382 of SEQ ID NO:2. In a mixture of CTLA4-Ig molecules, monomers having lysine at the C-terminus (residue 383 of SEQ ID NO:2) can be present, for example, at about 4-5%.

A CTLA4-Ig monomer molecule can comprise an extracellular domain of human CTLA4. In one embodiment, the extracellular domain can comprise the nucleotide sequence of nucleotides 89-463 of SEQ ID NO:1 that code for amino acids 27-151 of SEQ ID NO:2. In another embodiment, the extracellular domain can comprise mutant sequences of human CTLA4. In another embodiment, the extracellular domain can comprise nucleotide changes to nucleotides 89-463 of SEQ ID NO:1 such that conservative amino acid changes are made. In another embodiment, the extracellular domain can comprise a nucleotide sequence that is at least 75%, 80%, 85%, 90%, 95%, 96%, 97%, 98%, or 99% identical to nucleotides 89-463 of SEQ ID NO:1.

A CTLA4-Ig monomer molecule can comprise a constant region of a human immunoglobulin. This constant region can be a portion of a constant region; this constant region can have a wild-type or mutant sequence. The constant region can be from human IgG1, IgG2, IgG3, IgG4, IgM, IgA1, IgA2, IgD or IgE. The constant region can be from a light chain or a heavy chain of an immunoglobulin. Where the constant region is from an IgG, IgD, or IgA molecule, the constant region can comprise one or more of the following constant region domains: CL, CH1, hinge, CH2, or CH3. Where the constant region is from IgM or IgE, the constant region can comprise one or more of the following constant region domains: CL, CH1, CH2, CH3, or Ca4. In one embodiment, the constant region can comprise on or more constant region domains from IgG, IgD, IgA, IgM or IgE.

In one embodiment, a CTLA4-Ig monomer molecule comprises a modified human IgG1 hinge region (nucleotides 464-508 of SEQ ID NO: 1; amino acids 152-166 of SEQ ID NO:2) wherein the serines at amino acid residues 156, 162, and 165 of SEQ ID NO:2 have been engineered from cysteines present in the wild-type sequence.

In one embodiment, a CTLA4-Ig monomer molecule comprises a modified human IgG1 CH2 region and a wild-type CH3 region (the modified human IgG1 CH2 domain having nucleotides 509-838 of SEQ ID NO: 1 and amino acids 167-276 of SEQ ID NO:2; the human IgG1 CH3 domain having nucleotides 839-1159 of SEQ ID NO:1 and amino acids 277-383 of SEQ ID NO:2).

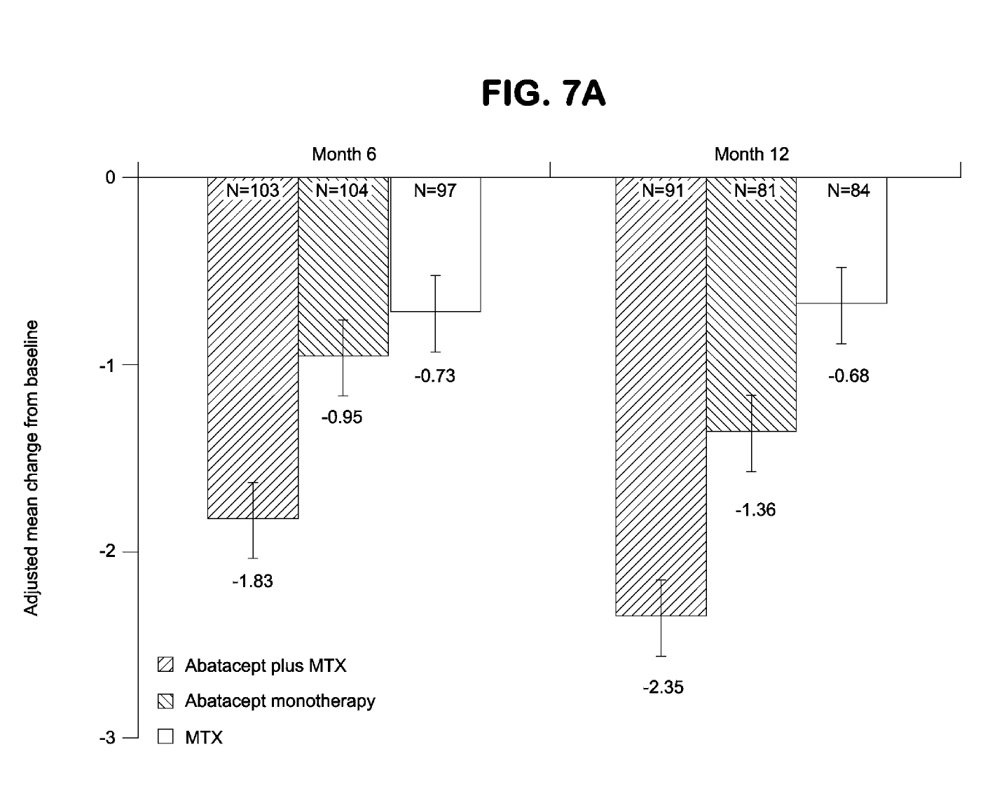

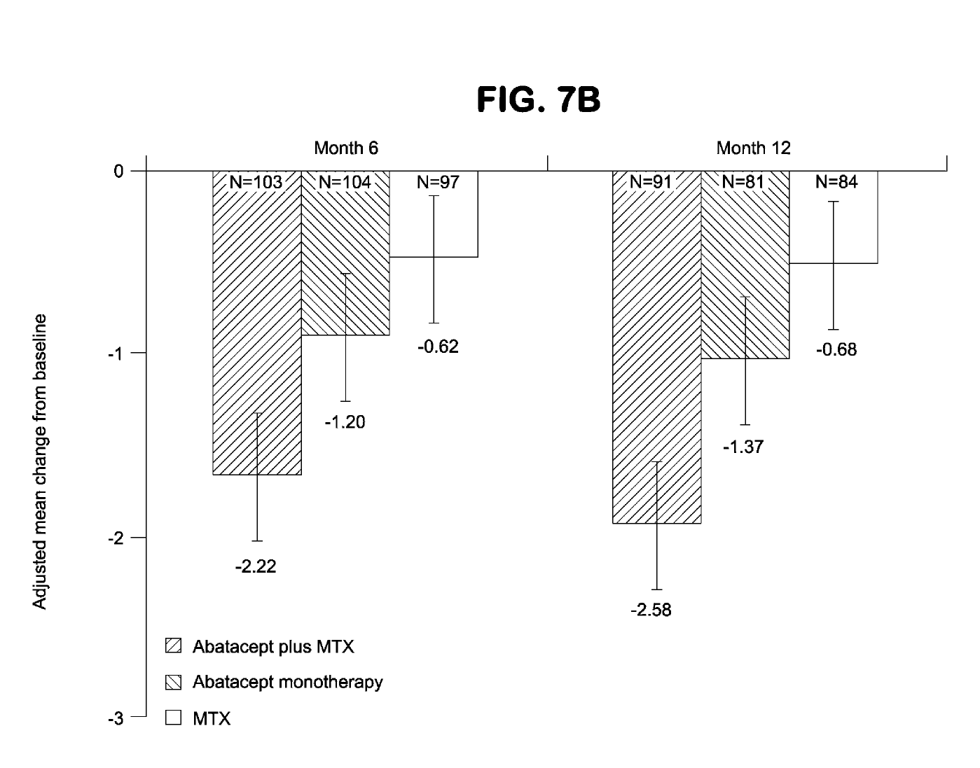

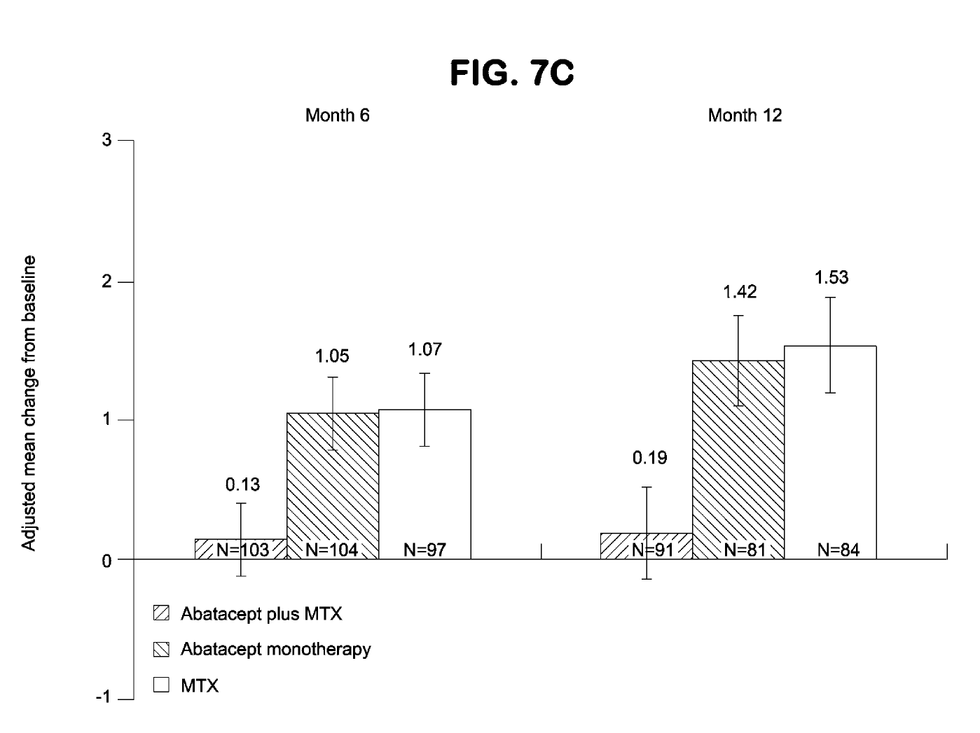

In one embodiment, a CTLA4-Ig molecule population comprises monomers having a sequence shown in any one or more of FIG. 7, 8, or 9 of the U.S. Pat. No. 7,094,874, issued on Aug. 22, 2006, and U.S. Pat. No. 7,455,835, issued on Nov. 25, 2008, which are hereby incorporated by reference in its entirety.

In one embodiment, a CTLA4-Ig tetramer molecule comprises two pairs or two dimers of CTLA4-Ig polypeptides, wherein each polypeptide has one of the following amino acid sequences: (i) 26-383 of SEQ ID NO:2, (ii) 26-382 of SEQ ID NO:2, (iii) 27-383 of SEQ ID NO:2, or (iv) 27-382 of SEQ ID NO:2, or optionally (v) 25-382 of SEQ ID NO:2, or (vi) 25-383 of SEQ ID NO:2. Each member of the pair of polypeptides or dimer is covalently linked to the other member, and the two pairs of polypeptides are non-covalently associated with one another thereby forming a tetramer. Such tetramer molecules are capable of binding to CD80 or CD86.

In another embodiment, such tetramer molecules can bind to CD80 or CD86 with an avidity that is at least 2-fold greater than the binding avidity of a CTLA4-Ig dimer (whose monomers have one of the above amino acid sequences) to CD80 or CD86. In another embodiment, such tetramer molecules can bind to CD80 or CD86 with an avidity that is at least 2-fold greater than the binding affinity or avidity of wild-type CTLA4 to CD80 or CD86. Such greater avidity can contribute to higher efficacy in treating immune disorders and other diseases as described below. In addition, greater or improved avidity can produce the result of higher potency of a drug. For example, a therapeutic composition comprising CTLA4-Ig tetramer would have a higher avidity and therefore higher potency than the same amount of a therapeutic composition having CTLA4-Ig monomer. In another embodiment, such tetramer molecules can have at least a 2-fold greater inhibition on T cell proliferation as compared to a CTLA4-Ig dimer (whose monomers have one of the above amino acid sequences). In another embodiment, such tetramer molecules can have at least a 2-fold greater inhibition on T cell proliferation as compared to a wild-type CTLA4 molecule.

T cell proliferation can be measured using standard assays known in the art. For example, one of the most common ways to assess T cell proliferation is to stimulate T cells via antigen or agonistic antibodies to TCR and to measure, for example, the incorporation of titrated thymidine (3H-TdR) in proliferating T cells or the amount of cytokines released by proliferating T cells into culture. The inhibitory effect of CTLA4-Ig molecules upon T cell activation or proliferation can thereby be measured.

### Methods for Producing the CTLA4Ig Molecules of the Invention

Expression of CTLA4Ig molecules can be in prokaryotic cells. Prokaryotes most frequently are represented by various strains of bacteria. The bacteria may be a gram positive or a gram negative. Typically, gram-negative bacteria such as E. coli are preferred. Other microbial strains may also be used.

Sequences, described above, encoding CTLA4Ig molecules can be inserted into a vector designed for expressing foreign sequences in prokaryotic cells such as E. coli. These vectors can include commonly used prokaryotic control sequences which are defined herein to include promoters for transcription initiation, optionally with an operator, along with ribosome binding site sequences, include such commonly used promoters as the beta-lactamase (penicillinase) and lactose (lac) promoter systems (Chang et al., Nature, 198:1056 (1977)), the tryptophan (trp) promoter system (Goeddel et al., Nucleic Acids Res., 8:4057 (1980)) and the lambda derived P_{L} promoter and N-gene ribosome binding site (Shimatake et al., Nature, 292:128 (1981)).

Such expression vectors will also include origins of replication and selectable markers, such as a beta-lactamase or neomycin phosphotransferase gene conferring resistance to antibiotics, so that the vectors can replicate in bacteria and cells carrying the plasmids can be selected for when grown in the presence of antibiotics, such as ampicillin or kanamycin.

The expression plasmid can be introduced into prokaryotic cells via a variety of standard methods, including but not limited to CaCl₂-shock (Cohen, Proc. Natl. Acad. Sci. USA, 69:2110 (1972), and Sambrook et al., eds., Molecular Cloning: A Laboratory Manual, Second Edition, Cold Spring Harbor Press (1989)) and electroporation.

In accordance with the practice of the invention, eukaryotic cells are also suitable host cells. Examples of eukaryotic cells include any animal cell, whether primary or immortalized, yeast (e.g., Saccharomyces cerevisiae, Schizosaccharomyces pombe, and Pichia pastoris), and plant cells. Myeloma, COS and CHO cells are examples of animal cells that may be used as hosts. Particular CHO cells include, but are not limited to, DG44 (Chasin et al., Som. Cell. Molec. Genet., 12:555-556 (1986); Kolkekar, Biochemistry, 36:10901-10909 (1997)), CHO-K1 (ATCC No. CCL-61), CHO-K1 Tet-On cell line (Clontech), CHO designated ECACC 85050302 (CAMR, Salisbury, Wiltshire, UK), CHO clone 13 (GEIMG, Genova, IT), CHO clone B (GEIMG, Genova, IT), CHO-K1/SF designated ECACC 93061607 (CAMR, Salisbury, Wiltshire, UK), and RR-CHOK1 designated ECACC 92052129 (CAMR, Salisbury, Wiltshire, UK). Illustrative plant cells include tobacco (whole plants, cell culture, or callus), corn, soybean, and rice cells. Corn, soybean, and rice seeds are also acceptable.

Nucleic acid sequences encoding CTLA4Ig molecules described above can also be inserted into a vector designed for expressing foreign sequences in a eukaryotic host. The regulatory elements of the vector can vary according to the particular eukaryotic host.

Commonly used eukaryotic control sequences for use in expression vectors include promoters and control sequences compatible with mammalian cells such as, for example, CMV promoter (CDM8 vector) and avian sarcoma virus (ASV) (nLN vector). Other commonly used promoters include the early and late promoters from Simian Virus 40 (SV40) (Fiers et al., Nature, 273:113 (1973)), or other viral promoters such as those derived from polyoma, Adenovirus 2, and bovine papilloma virus. An inducible promoter, such as hMTII (Karin et al., Nature, 299:797-802 (1982)) may also be used.

Vectors for expressing CTLA4Ig molecules in eukaryotes may also carry sequences called enhancer regions. These are important in optimizing gene expression and are found either upstream or downstream of the promoter region.

Examples of expression vectors for eukaryotic host cells include, but are not limited to, vectors for mammalian host cells (e.g., BPV-1, pHyg, pRSV, pSV2, pTK2 (Maniatis); pIRES (Clontech); pRc/CMV2, pRc/RSV, pSFV1 (Life Technologies); pVPakc Vectors, pCMV vectors, pSG5 vectors (Stratagene)), retroviral vectors (e.g., pFB vectors (Stratagene)), pCDNA-3 (Invitrogen) or modified forms thereof, adenoviral vectors; Adeno-associated virus vectors, baculovirus vectors, yeast vectors (e.g., pESC vectors (Stratagene)).

Nucleic acid sequences encodingCTLA4Ig molecules can integrate into the genome of the eukaryotic host cell and replicate as the host genome replicates. Alternatively, the vector carrying CTLA4Ig molecules can contain origins of replication allowing for extrachromosomal replication.

For expressing the nucleic acid sequences in Saccharomyces cerevisiae, the origin of replication from the endogenous yeast plasmid, the 2μ circle can be used. (Broach, Meth. Enzymol., 101:307 (1983)). Alternatively, sequences from the yeast genome capable of promoting autonomous replication can be used (see, for example, Stinchcomb et al., Nature, 282:39 (1979)); Tschemper et al., Gene, 10:157 (1980); and Clarke et al., Meth. Enzymol., 101:300 (1983)).

Transcriptional control sequences for yeast vectors include promoters for the synthesis of glycolytic enzymes (Hess et al., J. Adv. Enzyme Reg., 7:149 (1968) and Holland et al., Biochemistry, 17:4900 (1978)). Additional promoters known in the art include the CMV promoter provided in the CDM8 vector (Toyama et al., FEBS, 268:217-221 (1990)); the promoter for 3-phosphoglycerate kinase (Hitzeman et al., J. Biol. Chem., 255:2073 (1980)), and those for other glycolytic enzymes.

Other promoters are inducible because they can be regulated by environmental stimuli or the growth medium of the cells. These inducible promoters include those from the genes for heat shock proteins, alcohol dehydrogenase 2, isocytochrome C, acid phosphatase, enzymes associated with nitrogen catabolism, and enzymes responsible for maltose and galactose utilization.

Regulatory sequences may also be placed at the 3′ end of the coding sequences. These sequences may act to stabilize messenger RNA. Such terminators are found in the 3′ untranslated region following the coding sequences in several yeast-derived and mammalian genes.

Illustrative vectors for plants and plant cells include, but are not limited to, Agrobacterium Tᵢ plasmids, cauliflower mosaic virus (CaMV), and tomato golden mosaic virus (TGMV).

Mammalian cells can be transformed by methods including but not limited to, transfection in the presence of calcium phosphate, microinjection, electroporation, or via transduction with viral vectors.

Methods for introducing foreign DNA sequences into plant and yeast genomes include (1) mechanical methods, such as microinjection of DNA into single cells or protoplasts, vortexing cells with glass beads in the presence of DNA, or shooting DNA-coated tungsten or gold spheres into cells or protoplasts; (2) introducing DNA by making cell membranes permeable to macromolecules through polyethylene glycol treatment or subjection to high voltage electrical pulses (electroporation); or (3) the use of liposomes (containing cDNA) which fuse to cell membranes.

U.S. Pat. Nos. 7,332,303 and 7,541,164 teach processes for the production of proteins of the invention, specifically recombinant glycoprotein products, by animal or mammalian cell cultures and are herein incorporated by reference.

Following the protein production phase of the cell culture process, CTLA4Ig molecules are recovered from the cell culture medium using techniques understood by one skilled in the art. In particular, the CTLA4Ig molecule is recovered from the culture medium as a secreted polypeptide.

The culture medium is initially centrifuged to remove cellular debris and particulates. The desired protein subsequently is purified from contaminant DNA, soluble proteins, and polypeptides, with the following non-limiting purification procedures well-established in the art: SDS-PAGE; ammonium sulfate precipitation; ethanol precipitation; fractionation on immunoaffinity or ion-exchange columns; reverse phase HPLC; chromatography on silica or on an anion-exchange resin such as QAE or DEAE; chromatofocusing; gel filtration using, for example, SEPHADEX® G-75 column; and protein A SEPHAROSE® columns to remove contaminants such as IgG. Addition of a protease inhibitor, such as phenyl methyl sulfonyl fluoride (PMSF), or a protease inhibitor cocktail mix also can be useful to inhibit proteolytic degradation during purification. A person skilled in the art will recognize that purification methods suitable for a protein of interest, for example a glycoprotein, can require alterations to account for changes in the character of the protein upon expression in recombinant cell culture.

Purification techniques and methods that select for the carbohydrate groups of the glycoprotein are also of utility within the context of the present invention. For example, such techniques include, HPLC or ion-exchange chromatography using cation- or anion-exchange resins, wherein the more basic or more acidic fraction is collected, depending on which carbohydrate is being selected for. Use of such techniques also can result in the concomitant removal of contaminants.

The purification method can further comprise additional steps that inactivate and/or remove viruses and/or retroviruses that might potentially be present in the cell culture medium of mammalian cell lines. A significant number of viral clearance steps are available, including but not limited to, treating with chaotropes such as urea or guanidine, detergents, additional ultrafiltration/diafiltration steps, conventional separation, such as ion-exchange or size exclusion chromatography, pH extremes, heat, proteases, organic solvents or any combination thereof.

The purified CTLA4Ig molecule require concentration and a buffer exchange prior to storage or further processing. A Pall Filtron TFF system may be used to concentrate and exchange the elution buffer from the previous purification column with the final buffer desired for the drug substance.

In one aspect, purified CTLA4Ig molecules, which have been concentrated and subjected to diafiltration step, can be filled into 2-L BIOTAINER® bottles, 50-L bioprocess bag or any other suitable vessel. CTLA4Ig molecules in such vessels can be stored for about 60 days at 2° C. to 8° C. prior to freezing. Extended storage of purified CTLA4Ig molecules at 2° C. to 8° C. may lead to an increase in the proportion of HMW species. Therefore, for long-term storage, CTLA4Ig molecules can be frozen at about −70° C. prior to storage and stored at a temperate of about −40° C. The freezing temperature can vary from about −50° C. to about −90° C. The freezing time can vary and largely depends on the volume of the vessel that contains CTLA4Ig molecules, and the number of vessels that are loaded in the freezer. For example, in one embodiment, CTLA4Ig molecules are in 2-L BIOTAINER® bottles. Loading of less than four 2-L BIOTAINER® bottles in the freezer may require from about 14 to at least 18 hours of freezing time. Loading of at least four bottles may require from about 18 to at least 24 hours of freezing time. Vessels with frozen CTLA4Ig molecules are stored at a temperature from about −35° C. to about −55° C. The storage time at a temperature of about −35° C. to about −55° C. can vary and can be as short as 18 hours. The frozen drug substance can be thawed in a control manner for formulation of drug product.

U.S. Publication No. 2009/0252749 teaches processes for the production of proteins of the invention, specifically recombinant glycoprotein products, by animal or mammalian cell cultures and is herein incorporated by reference.

### Pharmaceutical Composition

The methods of the present invention utilizes pharmaceutical compositions comprising the CTLA4Ig molecules admixed with an acceptable carrier or adjuvant which is known to those of skill of the art. The pharmaceutical compositions preferably include suitable carriers and adjuvants which include any material which when combined with the CTLA4Ig molecule retains the molecule's activity and is non-reactive with the subject's immune system. These carriers and adjuvants include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, phosphate buffered saline solution, water, emulsions (e.g., oil/water emulsion), salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances and polyethylene glycol. Other carriers may also include sterile solutions; tablets, including coated tablets and capsules. Typically such carriers contain excipients such as starch, milk, sugar (e.g., sucrose, glucose, maltose), certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods. Such compositions may also be formulated within various lipid compositions, such as, for example, liposomes as well as in various polymeric compositions, such as polymer microspheres.

Formulations comprising soluble CTLA4 molecules are described in U.S. Pat. No. 8,476,239 and are hereby incorporated by reference into this application. As described in U.S. Pat. No. 8,476,239, soluble CTLA4 molecules may be formulated for IV and subcutaneous applications. Briefly, a suitable subcutaneous (SC) formulation comprises CTLA4Ig molecules at a protein concentration of at least 100 mg/ml in combination with a sugar at stabilizing levels in an aqueous carrier.

An example of a CTLA4Ig SC drug product that is delivered via a pre-filed syringe utilized in the method of the invention described in Example III is provided in Table 2 below.

TABLE 2
Composition of CTLA4Ig SC Drug Product,
125 mg/ml (125 mg/syringe)

| Component | Amount (mg/syringe) |
|---|---|
| CTLA4Ig | 125 |
| Sucrose | 170 |
| Poloxamer 188 | 8.0 |
| Sodium phosphate monobasic, monohydrate | 0.143 |
| Sodium phosphate dibasic, anhydrous | 0.971 |
| Water for Injection | q.s. to 1. ml |

Examples I and II of the instant specification describe the manufacture of an intravenous (IV) and subcutaneous formulation of CTLA4Ig useful in the methods of the invention.

TABLE 3
Composition of Lyophilized CTLA4Ig (250 mg/vial) Drug Product

| Component | Amount (mg/vial)^{a} |
|---|---|
| CTLA4Ig | 262.5 |
| Maltose monohydrate | 525 |
| Sodium phosphate monobasic, monohydrate^{b} | 18.1 |
| Sodium chloride^{b} | 15.3 |
| Hydrochloric Acid | Adjust to 7.5 |
| Sodium hydroxide | Adjust to 7.5 |

^{a}Includes a 5% overfill for vial, needle, syringe loss.
^{b}These components are present in the CTLA4Ig drug substance solution.

The lyophilized drug product may be constituted with an aqueous carrier. The aqueous carrier of interest herein is one which is pharmaceutically acceptable (safe and non-toxic for administration to a human) and is useful for the preparation of a liquid formulation, after lyophilization. Typically, the lyophilized drug product is constituted to about 25 mg/ml with 10 ml of either Sterile Water for Injection, USP (SWFI) or 0.9% Sodium Chloride Injection, USP. The constituted solution is further diluted to drug product concentrations between 1 and 10 mg/ml with 0.9% Sodium Chloride Injection, USP. The diluted drug product for injection is isotonic and suitable for administration by intravenous infusion.

### Methods of Use

The present invention provides a method of achieving drug-free remission in subjects with early RA comprising administering to the subject in need thereof an effective amount of the CTLA4 molecule or pharmaceutical composition thereof.

The methods of the invention also may be used to inhibit structural damage of the joints in subjects with early RA as assessed by erosion and bone marrow edema scoring and/or synovitis scoring of the wrist and hand.

The amount of symptom relief provided by the present invention can be measured using any of the accepted criteria established to measure and document symptom relief in a clinical setting. Acceptable criteria for measuring symptom relief may include scores based on the criteria established by the American College of Rheumatology (e.g., ACR 20), the four measures of symptom relief (in: “CDER Guideline for the Clinical Evaluation of Anti-Inflammatory and Antirheumatic Drugs—FDA 1988”), and the Health Assessment Questionnaire (HAQ) (Fries, J. F. et al., J. Rheumatol., 9:789-793 (1982)). For a general description of these criteria, see Guidance for Industry: Clinical Development Programs for Drugs, Devices, and Biological products for the Treatment of Rheumatoid Arthritis (RA) (February 1999).

The present invention provides various methods, local or systemic, for administering the CTLA4Ig molecule alone or in conjunction with other therapeutic drugs. The methods include intravenous, intramuscular, intraperitoneal, oral, inhalation and subcutaneous methods, as well as implantable pump, continuous infusion, gene therapy, liposomes, suppositories, topical contact, vesicles, capsules and injection methods.

The CTLA4Ig, compounded with a carrier, is commonly lyophilized for storage and is reconstituted with water or a buffered solution prior to administration (see Example I). As is standard practice in the art, the compositions of the invention may be administered to the subject in any pharmaceutically acceptable form.

The CTLA4Ig compounded with a carrier may be provided as a subcutaneous formulation ready for administration (see Example II). The subcutaneous formulation may be provided in a vial or prefilled syringe.

The most effective mode of administration and dosage regimen for the formulations of this invention depends upon the patient's health and response to treatment and the judgment of the treating physician. In accordance with the practice of the invention an effective amount for treating a subject is an amount about 0.1 to 100 mg/kg weight of a subject. In another embodiment, the effective amount is an amount about 0.1 to 20 mg/kg weight of a subject, preferably 1 to 10 mg/kg weight of a subject. In a specific embodiment, the effective amount of CTLA4Ig is about 2 mg/kg weight of a subject. In another specific embodiment, the effective amount of CTLA4Ig is about 10 mg/kg weight of a subject. In another specific embodiment, an effective amount of CTLA4Ig is 500 mg for a subject weighing less than 60 kg, 750 mg for a subject weighing between 60-100 kg and 1000 mg for a subject weighing more than 100 kg. In another embodiment, the effective amount of CTLA4Ig is 125 mg administered subcutaneously on a weekly basis.

The CTLA4Ig molecule formulations of the invention may be administered to a subject in an amount and for a time (e.g., length of time and/or multiple times) sufficient to block endogenous B7 (e.g., CD80 and/or CD86) molecules from binding their respective ligands, in the subject. Blockage of endogenous B7/ligand binding thereby inhibits interactions between B7-positive cells (e.g., CD80- and/or CD86-positive cells) with CD28- and/or CTLA4-positive cells. Accordingly, dosages of the agents can vary depending on the subject and the mode of administration.

An effective amount of CTLA4Ig molecule may be administered to a subject daily, weekly, monthly and/or yearly, in single or multiple times per hour/day/week/month/year, depending on need. For example, in one embodiment, an effective amount of the CTLA4Ig molecule may initially be administered once every two weeks for a month, and then once every month thereafter or Days 1, 15, 29 and monthly thereafter. A +/−3 day window is allowed for earlier doses (i.e., Days 15 and 29). A +/−7 day window is allowed for the monthly doses thereafter.

Alternatively, one knowledgeable in the art would be able to modify the administration regimen in response to the patients risk status and/or response to the therapy. For example, the regimen described above could be modified by adding administration day 5 to the regimen.

As used herein, “four weeks”, “month”, “months” or “monthly” refers to a period of 28±7 days

Typically, doses of the CTLA4Ig molecule formulation of the invention are based on body weight, and administration regimens may be dictated by the target serum trough profiles. Typically, target trough serum concentration of CTLA4Ig molecules of the invention between about 3 μg/mL and about 35 μg/mL will be sufficient to treat RA or achieve remission in subjects with RA, preferably between about 5 μg/mL and about 30 μg/mL, more preferably between about 10 μg/mL and about 30 μg/mL. One knowledgeable in the art would be able to adjust the dosage and/or administration schedule of CTLA4Ig to achieve the desired serum trough concentrations.

The administration of the molecules or pharmaceutical compositions of the invention can be via a 30 minute to one or more hour intravenous infusion. Alternatively, single to multiple subcutaneous injections can deliver the required dosage.

The CTLA4Ig molecules of the invention may be administered concomitantly or sequentially in conjunction with other immunosuppressive/immunomodulatory therapy, e.g., as herein specified, dosages of the co-administered immunosuppressant, or immunomodulatory compound will of course vary depending on the type of co-drug employed.

Non-steroidal anti-inflammatory drugs (NSAIDs) may be administered in concomitantly or sequentially in conjunction with the CTLA4Ig molecule of the invention. NSAIDs reduce inflammatory reactions in a subject. NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, meloxicam and tramadol.

Corticosteroids may be administered concomitantly or sequentially in conjunction with the CTLA4Ig molecule of the invention. For example, stable low dose oral corticosteroid (equivalent to ≦10 mg prednisone daily), or high dose corticosteroids administered every six months as an oral course (equivalent to 20 mg/day prednisone daily for a maximum of two weeks), or a single IM (intramuscular) dose or a single IA (intra-articular) dose.

Examples of corticosteroids include but are not limited to, betamethasone, budesonide, cortisol, cortisone, dexamethasone, hydrocritisone, methylprednisolone, prednisolone, prednisone and triamcinolone.

Typically, the standard dosages and administration regimen of the co-administered drugs described above are not influenced by the addition of the CTLA4Ig molecules of the invention to the treatment regimen. However, one knowledgeable in the art may prescribe lower doses of the co-administered drugs due to the incorporation of the less toxic CTLA4Ig molecules of the invention into the treatment regimen. Prescribing information may be based on the package insert for each co-administered drug.

Method of Achieving Drug-Free Remission in Subjects with Early RA

As discussed previously, joint destruction occurs early in RA. This insight has highlighted the need for therapies which can fundamentally alter and not merely suppress the inflammatory processes which cause debilitating symptoms and structural damage early on in the course of RA. Consequently, this has placed increasing emphasis on earlier diagnosis and treatment of RA.

Further, identifying early RA subjects with poor prognosis, who therefore would be ideal candidates for targeted therapy aimed at the underlying mechanisms driving inflammation and joint destruction in RA is key to achieving drug-free remission. Such an approach would prevent the development of joint damage, functional disability and subsequent impaired quality of life that unfortunately characterizes the natural history of RA.

One embodiment of the invention is a method of identifying subjects likely to achieve drug-free remission. The subjects with early RA who are likely to achieve sustained drug-free remission have highly active disease and poor prognostic markers. For example, the early RA subject likely to achieve drug-free remission is characterized as having active clinical synovitis of ≧2 joints for ≧8 weeks, DAS28 (CRP) ≧3.2 and anti-citrullinated peptide (CCP)-2 antibody positivity.

The method of the invention further comprises administration of an effective amount of CTLA4 molecule or pharmaceutical composition thereof to the early RA subject with highly active disease and poor prognostic markers. For example, CTLA4Ig can be administered at 125 mg/week subcutaneously or 10 mg/kg weight of a subject biweekly, monthly or in a schedule sufficient to meet a target trough serum concentration of between about 5 μg/mL and about 30 μg/mL.

The CTLA4 molecule is administered to the early RA subjects with highly active disease and poor prognostic markers at the above referenced dose until remission is achieved. In the method of the invention remission is DAS-defined remission (Disease Activity Score Calculator for Rheumatoid Arthritis (C-reactive protein) [DAS28(CRP)]) <2.6. DAS-defined remission is typically achieved by 12 months of CTLA4 administration.

The method of the invention further comprises withdrawal of all immunosuppressive/immunomodulatory therapy once DAS-defined remission is achieved. One or more of the RA medications may be withdrawn rapidly or tapered off over a period of time, such as 1 month, 2 months or 3 months. For example, the CTLA4 molecule is rapidly withdrawn, while the other RA medications are tapered off over a period of time, such as 1 month, 2, months or 3 months.

Another embodiment of the invention is a method of inhibiting structural damage in subjects with early RA by achieving drug-free remission. In the method of the invention structural damage as assessed by erosion, osteitis and/or synovitis scoring of the wrist and hand.

Example III shows that radiographic changes measured by MRI in each of the treatment groups were consistent with clinical efficacy outcomes. Abatacept plus MTX and abatacept monotherapy resulted in numerically greater decreases from baseline in synovitis and osteitis scores, and abatacept plus MTX resulted in less progression of erosion score, than MTX at 12 months (see FIG. 7A-C).

Example III describes the first clinical study (AVERT) to demonstrate that remission can be maintained after rapid withdrawal of all therapy (including csDMARDs, biologic DMARDS and corticosteroids) in patients with early RA receiving abatacept(i.e., CTLA4-Ig). Patients treated with abatacept achieved significantly higher rates of DAS-defined remission than MTX on-treatment; and, a small but significantly higher number of patients achieved sustained absolute, drug-free, DAS-defined remission following withdrawal of all RA treatment. These results support the hypothesis that early treatment with a T-cell immunomodulator that acts high up in the inflammatory cascade can increase drug-free remission.

In AVERT, patients had highly active disease and poor prognostic markers, a combination associated with enhanced probability of joint damage and disease progression. Abatacept plus MTX achieved robust efficacy versus MTX, as demonstrated by multiple measures of remission and HAQ-DI, and consistent structural benefits.

AVERT provides a large dataset assessing abatacept monotherapy, which is of interest because many patients cannot tolerate MTX. A similar number of patients receiving abatacept achieved DAS-defined remission versus MTX at Month 12, but the overall data showed that abatacept monotherapy had numerically higher benefit compared with MTX. The MRI findings for abatacept monotherapy also demonstrated a numerically greater benefit on osteitis and synovitis compared with MTX alone at Month 12.

Following withdrawal of all therapy, a small but significant number of patients sustained drug-free remission following prior treatment with abatacept plus MTX compared with MTX alone. The data indicate that, with abatacept plus MTX treatment, one in four patients was able to maintain drug-free remission through 6 months. This effect is not a consequence of the half-life of abatacept (14.3 days) as assessments were performed up to 6 months after the withdrawal of all treatment (>5 half-lives). Moreover, the post hoc analyses of the patients that sustained drug-free remission indicate that patients with shorter symptom duration and lower disease activity at baseline, or longer sustained DAS-defined remission prior to treatment withdrawal, were more likely to maintain drug-free remission. These associations were observed specifically in both abatacept arms, suggesting a biologic effect was responsible.

These data therefore indicate that patients with early RA, with a very short symptom duration and milder disease activity, are able to achieve sustained and complete drug-free remission following treatment with abatacept.

The DAS-defined remission cut-off of <2.6, although corresponding to the American Rheumatology Association definition of clinical remission in RA (Fransen, J. et al., “Remission in rheumatoid arthritis: agreement of the disease activity score (DAS28) with the ARA preliminary remission criteria”, Rheumatology (Oxford), 43:1252-1255 (2004)), has now been replaced with other measures of remission (Felson, D. T. et al., “American College of Rheumatology/European League against Rheumatism provisional definition of remission in rheumatoid arthritis for clinical trials”, Ann. Rheum. Dis., 70:404-413 (2011)); such as SDAI and Boolean which are also reported here. The cut-off is based on erythrocyte sedimentation rate (ESR), and a CRP cut-off has yet to be defined. In AVERT, CRP was interchanged with ESR to reduce the variability of the acute phase reactant and aid standardization across study centers. Data are obtained from patients with early RA with active disease and poor prognostic factors, which limit its generalizability to the overall RA population. The withdrawal analyses were limited by the small number of patients who remained in the withdrawal period. The gradual tapering of RA medication may result in higher remission rates than the rapid withdrawal of all RA therapy applied in AVERT and will be assessed in other trials.

AVERT establishes the benefit of abatacept treatment in combination with MTX in an early RA population and further indicates that, in early RA, drug-free remission is possible following treatment with abatacept. The novel achievement of sustained remission following withdrawal of all RA therapy is suggestive of an underlying effect of abatacept's mechanism on autoimmune processes. A withdrawal treatment strategy is a highly desirable goal for patients and physicians in the long-term treatment of RA. Treat-to-remission is now a well accepted goal of RA therapy.

The invention will be more fully understood by reference to the following examples. They should not, however, be construed as limiting the scope of the invention. All citations throughout the disclosure are hereby expressly incorporated by reference.

### Example I

CTLA4Ig, lyophilized, (250 mg/vial) drug product is a sterile, non-pyrogenic lyophile suitable for intravenous (IV) administration. Each single-use vial contains 250 mg of CTLA4Ig which is constituted with Sterile Water for Injection, USP and further diluted with 0.9% Sodium Chloride Injection, USP, at the time of use.

The batch formula for a 115 liter batch size is described in Table 4 below.

TABLE 4
Batch Formula

| Component | Amount (kg) |
|---|---|
| CTLA4Ig drug substance^{a} | 4.6 |
| Maltose monohydrate | 9.2 |
| Hydrochloric Acid | Adjust to pH 7.5 |
| Sodium hydroxide | Adjust to pH 7.5 |
| Water for Injection | q.s. to 119.6^{b} |

^{a}CTLA4Ig drug substance: protein concentration 50 mg/ml, 25 mM sodium phosphate, 50 mM sodium chloride, pH of 7.5, <5% HMW species.
^{b}Formulated bulk solution density = approx. 1.04 g/ml.

The required quantity of CTLA4Ig drug substance is added to a cleaned and sterilized stainless steel compounding vessel equipped with a mixer. The drug substance solution is mixed at 250±50 rpm while maintaining the solution temperature between 5° C.-25° C.

The required quantity of maltose monohydrate powder is added to the compounding vessel. The solution is mixed for a minimum of 10 minutes at 15° C.-25° C.

The solution pH is adjusted to 7.3-7.7, if necessary using the previously prepared 1 N sodium hydroxide solution or 1 N hydrochloric acid solution. The batch is brought to the final batch weight (final q.s.) using Water for Injection, USP, and mixed for a minimum of 8 minutes. The formulated bulk solution is sampled for pH.

Formulated Bulk Solution is pre-filtered with one 0.45-μm filter. The formulated bulk solution after 0.45-μm filter is sampled for bioburden and bacterial endotoxin (BET).

The pre-filtered formulated bulk Solution is sterile filtered with two 0.22-μm filters in series prior to filling.

Sterile filtered Formulated Bulk Solution is filled and partially stoppered with a 20 nm-Daikyo gray butyl stopper by a fully automatic filling/stoppering machine. The 15-cc Type I flint tubing glass vials are washed and sterilized/depyrogenated.

The filled and partially stoppered drug product vials are lyophilized. A summary of the freeze drying cycle used during lyophilization of CTLA4Ig drug product is provided in Table 5 below.

TABLE 5
Freeze Dry Cycle for CTLA4Ig Lyophilized Drug Product

| Process parameter | In-process control |
|---|---|
| Loading Temperature | 5 ± 3° C. |
| Freezing (Shelf Ramp) | From 5° C. to −45° C. in 2.5 hr. |
| Freezing | Hold at −45 ± 3° C. for 4 hr. |
| Primary Drying (Shelf Ramp) | From −45° C. to −19° C. in 2 hr. |
| Primary Drying (Vacuum) | 100 ± 20 microns |
| Primary Drying | Hold at −19 ± 2° C. for 84 hr. |
| Intermediate Drying (Shelf Ramp) | From −19° C. to 0° C. in 2 hr. |
| Intermediate Drying | Hold at 0 ± 3° C. for 8 hr. |
| Secondary Drying (Shelf Ramp) | From 0° C. to 30° C. in 2.5 hr. |
| Secondary Drying (Vacuum) | 100 ± 20 microns |
| Secondary Drying | Hold at 30° C. for 12 hr. |
| Stoppering | 30 ± 3° C. |
| Stoppering (Vacuum) | 500 ± 100 microns |
| Storage Before Unloading | Hold at 20 ± 3° C. for at least 4 hr. |

At the end of the lyophilization cycle, the chamber pressure is raised to 500 microns using sterile filtered nitrogen and vial stoppering is performed under vacuum. The stoppered vials remain inside the lyophilizer for at least 4 hours. The lyophilized and stoppered vials are sealed with a 20-mm aluminum, white flip-off seal under HEPA filtered air by the capping machine. The sealed vials are rinsed with deionized water by an exterior vial washer. The washed drug product vials are stored at 2° C. to 8° C.

The composition of lyophilized CTLA4Ig (250 mg/vial) drug product is listed in Table 6 below.

TABLE 6
Composition of Lyophilized CTLA4Ig (250 mg/vial) Drug Product

| Component | Amount (mg/vial)^{b} |
|---|---|
| CTLA4Ig | 262.5 |
| Maltose monohydrate | 525 |
| Sodium phosphate monobasic, monohydrate^{b} | 18.1 |
| Sodium chloride^{b} | 15.3 |
| Hydrochloric Acid | Adjust to 7.5 |
| Sodium hydroxide | Adjust to 7.5 |

^{a} Includes a 5% overfill for vial, needle, syringe loss.
^{b}These components are present in the CTLA4Ig drug substance solution.

### Example II

CTLA4Ig SC, 125 mg/ml (125 mg/vial) drug product is formulated as a sterile, non-pyrogenic ready-to-use solution suitable for subcutaneous administration. A batch of CTLA4Ig SC, 125 mg/ml (125 mg/vial) drug product is manufactured at 5-L scale (3,500 vials). The batch formula is described in Table 7 below.

TABLE 7
Batch Formula

| Component | Amount (gm) |
|---|---|
| CTLA4Ig drug substance^{a} | 625 |
| Sucrose | 850 |
| Poloxamer 188 | 40 |
| Sodium phosphate monobasic, monohydrate | 0.715 |
| Sodium phosphate dibasic, anhydrous | 4.86 |
| Water for Injection | q.s. to 5.0 L |
| Total Batch size (L) | 5.0 |

^{a}CTLA4Ig drug substance: protein concentration 50 mg/ml, 25 mM sodium phosphate, 50 mM sodium chloride, pH of 7.5, <5% HMW species.

As described above in Example I, the manufacturing process for CTLA4Ig SC, 125 mg/ml (125 mg/vial) drug product involves buffer exchange of the bulk drug substance from 25 mM sodium phosphate, 50 mM sodium chloride at a pH of 7.5 to 10 mM sodium phosphate pH 7.8 buffer, followed by concentration of the protein from ˜50 mg/ml to ˜150 mg/ml by removal of buffer. Sucrose and Poloxamer 188 are then dissolved in the concentrated protein solution and final batch weight is adjusted with 10 mM sodium phosphate buffer, pH 7.8. The bulk solution is filtered through 0.22 micron sterilizing filter and filled into sterilized and depyrogenated 5-cc Type I flint glass vials, stoppered with 20 mm rubber stoppers and sealed with 20 mm aluminum flip-off seals.

The composition of CTLA4Ig SC drug product, 125 mg/ml (125 mg/vial) is provided in Table 8 below.

TABLE 8
Composition of CTLA4Ig SC, 125 mg/ml
(125 mg/vial) Drug Product

| Component | Amount (mg/vial)^{c} |
|---|---|
| CTLA4Ig | 175 |
| Sucrose | 238 |
| Poloxamer 188 | 11.2 |
| Sodium phosphate monobasic, monohydrate | 0.20 |
| Sodium phosphate dibasic, anhydrous | 1.36 |
| Water for Injection | q.s. to 1.4 ml |

^{c}Includes 40% overfill for Vial, Needle, Syringe loss.

### Example III

Assessing Very Early Rheumatoid Arthritis Treatment (AVERT) was a phase 3b, randomized, active-controlled trial of 24 months, with a 12-month, double-blind treatment period.

### Study Design

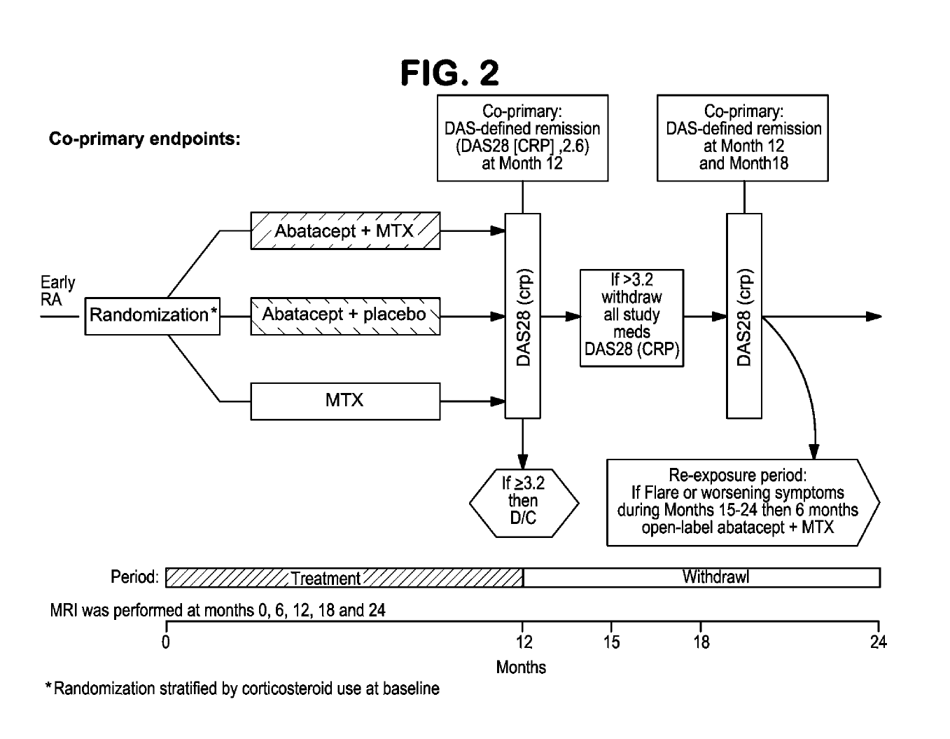

The study design is described graphically in FIG. 2.

### Inclusion Criteria

Willing to participate in the study and provided signed informed consent
Active clinical synovitis of ≧2 joints (including ≧1 small joint and not including distal interphalangeal joints), for ≧8 weeks at screening
Onset of persistent symptoms ≦2 years prior to screening
Disease Activity Score 28 (DAS28) C-reactive protein (CRP) ≧3.2 at screening
Anti-cyclic citrullinated peptide-2 positive
methotrexate (MTX) naïve or MTX ≦10 mg/kg for ≦4 weeks and no dose for 1 month prior to screening
Biologic naïve
Chloroquin, hydroxychloroquine and sulfasalazine stopped for ≧28 days (if received)
Stable dose oral corticosteroids (≦10 mg prednisone equivalent for ≧4 weeks) or intramuscular, intravenous or intra-articular corticosteroids ≧4 weeks prior to randomization (if received)
Age ≧18 years
Men and women of childbearing potential using an acceptable method of contraception to avoid pregnancy for up to 10 weeks (14 weeks in European Union) after last dose of study medication
Women with negative serum or urine pregnancy test within 48 hours prior to the start of investigational product
Women must not be breastfeeding
Investigators should follow the manufacturer's recommendations for MTX
Able to receive an magnetic resonance imaging (MRI)

### Exclusion Criteria

Met the diagnostic criteria for another rheumatic disease
Impaired, incapacitated or incapable of completing study-related assessments
Current symptoms of severe, progressive, or uncontrolled renal, hepatic, hematological, gastrointestinal, pulmonary, cardiac, neurological, or cerebral disease
Concomitant medical conditions that, in the opinion of the investigator, might place the patient at unacceptable risk for participation in this study
Women with a breast cancer screening study that is suspicious for malignancy, and in whom the possibility of malignancy cannot be reasonably excluded following additional clinical, laboratory or other diagnostic evaluations
History of cancer within the last 5 years (other than non-melanoma skin cell cancers cured by local resection). Existing non-melanoma skin cell cancers must be removed prior to dosing. Patients with carcinoma in situ, treated with definitive surgical intervention prior to study entry, were allowed
Clinically significant drug or alcohol abuse
Any serious acute bacterial infection (unless treated and completely resolved with antibiotics
Severe chronic or recurrent bacterial infections
Risk for TB: current clinical, radiographic or laboratory evidence of tuberculosis (TB); history of active TB ≦3 years ago; history of active TB >3 years ago unless documentation to support appropriate duration and type of prior anti-TB treatment; latent TB which was not successfully treated (unless active TB infection ruled out and treatment for latent TB with isoniazid for ≧4 weeks prior to dosing of study drug and negative chest radiograph at enrollment)
Herpes zoster resolved <2 months prior to enrollment
Evidence of active or latent bacterial or viral infections at time of potential enrollment
Hepatitis B surface antigen-positivity
Hepatitis C antibody-positivity and recombinant immunoblot assay positivity (RIBA-positivity) or polymerase chain reaction positivity (PCR positivity)
Hemoglobin <8.5 g/dL
White blood cells <3000/mm³
Platelets <100,000/mm³
Serum creatinine, alanine aminotransferase (ALT) or aspartate aminotransferase (AST) >2 times upper limit of normal
Any other laboratory test result that, in the opinion of the study investigator, might place the patient at unacceptable risk for participation in the study
Prior exposure to abatacept
Exposure to any investigational drug within 4 weeks or 5 half-lives, whichever is longer.
Currently receiving (or in the last 3 months) azathioprine, gold, leflunomide, immunoadsorption columns, mycophenylate mofetil, cyclosporine, other calceineurin inhibitors or D-Penicillamine
Intramuscular, intravenous or intra-articular corticosteroids ≦4 weeks prior to randomization
Sexually active fertile men not using effective birth control if partners are women of child-bearing potential
Prisoners or patients who are involuntarily incarcerated
Compulsorily detained for treatment of either a psychiatric or physical illness illiterate

The study population included adults (≧18 years old) with active clinical synovitis of ≧2 joints for ≧8 weeks, with persistent symptoms for ≦2 years; Disease Activity Score (DAS)28 (C-reactive protein [CRP]) ≧3.2 and anti-citrullinated peptide (CCP)-2 antibody positivity. Patients were MTX naïve or received MTX (≦10 mg/week) for ≦4 weeks with no MTX for 1 month prior to enrollment. Patients receiving oral corticosteroids were required to be on a stable dose (≦10 mg/day for ≧4 weeks) at initiation and to maintain that dose until Month 12.

In the 12-month treatment period, patients were randomized (1:1:1) to abatacept (i.e., CTLA4-Ig) plus MTX, abatacept monotherapy or MTX, stratified by corticosteroid use at baseline (yes/no) using a Centralized Randomization System. SC abatacept was administered at 125 mg/week. MTX was initiated at 7.5 mg/week and titrated to 15-20 mg/week within 6-8 weeks (≦10 mg/week permitted in patients with intolerance). All patients received concomitant folic acid therapy.

Patients with DAS28 (CRP)<2.6 at Month 12 could enter the 12-month withdrawal period during which all treatment was stopped: abatacept immediately and MTX and steroids tapered over 1 month. Patients with DAS28 (CRP) ≧3.2 were discontinued.

After Month 15, patients in the withdrawal period who experienced a flare of RA defined as two of the following: doubling of tender and swollen joint counts relative to Month 12, increase in DAS28 (CRP) ≧1.2 from Month 12, or investigator's judgment of RA flare, were eligible to enter a re-exposure period with open-label SC abatacept 125 mg plus MTX.

All patients underwent contrast magnetic resonance imaging (MRI) of the wrist and hand of the major affected upper limb at baseline and at 6, 12, 18 and 24 months.

### Outcome Measures

For the purpose of this study, DAS-defined remission was DAS28 (CRP)<2.6. Co-primary endpoints were: the proportion of randomized and treated patients in DAS-defined remission at (i) Month 12 and (ii) Months 12 and 18 for abatacept plus MTX versus MTX.

Secondary endpoints included: DAS-defined remission at (i) Month 12 and (ii) both Months 12 and 18 for abatacept monotherapy versus MTX; Health Assessment Questionnaire-Disability Index (HAQ-DI) response (≧0.3 points reduction from baseline); osteitis, synovitis and erosion score by MRI; safety and tolerability.

Exploratory endpoints included: Other remission rates (Simplified Disease Activity Index [SDAI; ≦3.3], Clinical Disease Activity Index [CDAI; ≦2.8] and Boolean remission [28-joint tender joint count ≦1 and 28-joint swollen joint count ≦1 and patient global assessment of disease activity [0-10 cm]≦1 and high-sensitivity CRP ≦1 mg/dL]), American College of Rheumatology (ACR) responses and Major Clinical Response (ACR 70 response for 6 months at any time period) in each arm.

### Statistical Analysis

A sample size of 116 patients per arm yielded 90% power to detect an expected difference of 22% for the first co-primary endpoint. This power estimate assumed that 60% of patients in the abatacept plus MTX arm and 38% of patients in the MTX arm would achieve DAS-defined remission (DAS28 [CRP]<2.6) at Month 12. Conditional on achieving the first co-primary endpoint, a sample size of 116 patients per arm yielded 98% power to detect an expected difference of 22% for the second co-primary endpoint. This power estimate assumed that 30% of patients in the abatacept plus MTX arm and 8% of patients in the MTX arm would achieve DAS-defined remission (DAS28 [CRP]<2.6) at both Months 12 and 18.

Co-primary endpoints were tested in hierarchical fashion. Odds ratios (with 95% confidence intervals [CI]) were calculated for abatacept plus MTX versus MTX using logistic regression adjusted for treatment group, corticosteroid use at baseline (yes/no) and baseline DAS28 (CRP); patients with missing baseline DAS28 (CRP) were not included. All patients who discontinued prior to completing the treatment or withdrawal period were imputed as non-responders for the Month 12 or 18 analyses.

Adjusted mean MRI change from baseline and standard error was calculated for all arms using a longitudinal repeated measures model. Safety assessments were based on the intent-to-treat population (patients who received ≧1 dose of study medication). Analysis of other secondary endpoints is described in the supplementary information.

### Statistical Analysis of Secondary and Exploratory Endpoints

Endpoints of Disease Activity Score (DAS)-defined remission, SDAI remission, CDAI remission, Boolean remission, ACR 20/50/70 response and Major Clinical Response over time were summarized using descriptive statistics (with 95% confidence intervals at each time point). Missing remission data not due to premature discontinuation and not at Day 1 of the treatment period or at Day 169 of the withdrawal period was imputed as a remission if the missing value occurred between two observed remissions. Missing ACR response data not due to premature discontinuation and not at Day 1 of the treatment period or at Day 169 of the withdrawal period was imputed as an ACR response if the missing value occurred between two observed ACR responses.

### Post Hoc Analyses

For each treatment arm, a post hoc analysis was performed of mean baseline characteristics for patients who achieved DAS-defined remission at only Month 12 and at both Months 12 and 18, and of the proportions of patients who achieved DAS-defined remission based on these characteristics. An analysis of the overall treatment effect in mean change from baseline in DAS28 (CRP) (including data up to Month 12 of the treatment period) was performed for each treatment arm. Overall treatment effect and treatment differences between the three arms were obtained using a longitudinal repeated measures model including fixed categorical effects of treatment, months, and prior corticosteroid use as well as the continuous fixed covariate of baseline value. An unstructured covariance matrix was used to represent the correlation of the repeated measures within each subject.

### Results

### Demographics and Baseline Characteristics

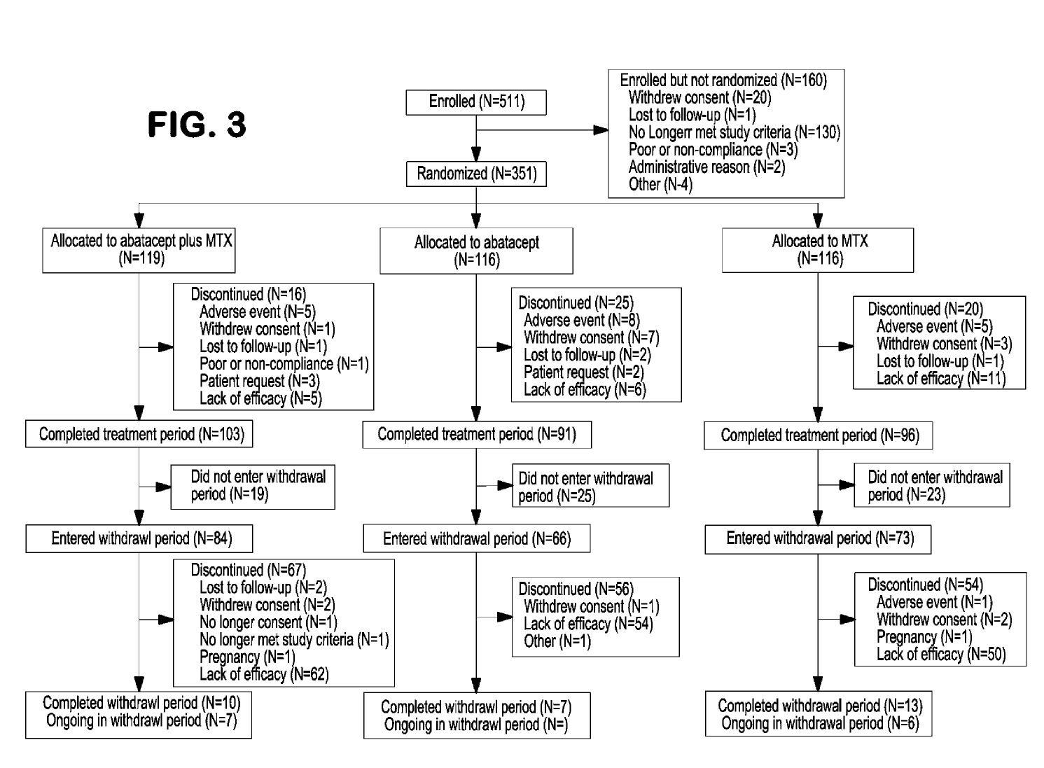

A total of 511 patients were enrolled and 351 patients at 72 worldwide sites were randomly assigned to treatment (abatacept plus MTX, n=119; abatacept monotherapy, n=116; MTX, n=116) (see FIG. 3)

As described in Table 9 below, patients had early RA (mean symptom duration 0.56 years) with highly inflammatory disease (mean tender joint count 23.3, swollen joint count 16.5 and CRP 17 mg/dL), severe disease activity (mean DAS28 [CRP] 5.44 and HAQ-DI 1.42) and poor prognostic factors (95.2% rheumatoid factor and anti-CCP-2 double positive).

TABLE 9
Demographics and Baseline Characteristics

| | Abatacept plus MTX | Abatacept Monotherapy | MTX | Total |
|---|---|---|---|---|
| Characteristic | (N = 119) | (N = 116) | (N = 116) | (N = 351) |
| Age - yr (median) | 46.4 ± 13.2 (45.0) | 45.4 ± 11.9 (45.0) | 49.1 ± 12.4 (49.0) | 47.0 ± 12.6 (47.0) |
| Weight - kg (median) | 73.0 ± 17.7 (68.7) | 72.1 ± 16.8 (69.5) | 74.1 ± 17.1 (71.5) | 73.1 ± 17.2 (69.9) |
| Female sex - no. (%) | 95 (79.8) | 89 (76.7) | 89 (76.7) | 273 (77.8) |
| White race - no. (%) | 100 (84.0) | 95 (81.9) | 102 (87.9) | 297 (84.6) |
| Geographic region - no. (%) | | | | |
| North America | 17 (14.3) | 21 (18.1) | 15 (12.9) | 53 (15.1) |
| South America | 26 (21.8) | 24 (20.7) | 25 (21.6) | 75 (21.4) |
| Europe | 47 (39.5) | 42 (36.2) | 48 (41.4) | 137 (39.0) |
| ROW | 29 (24.4) | 29 (25.0) | 28 (24.1) | 86 (24.5) |
| RA symptom duration - yr | 0.58 ± 0.50 | 0.59 ± 0.52 | 0.50 ± 0.49 | 0.56 ± 0.50 |
| RF positive - no. (%) | 113 (95.0) | 111 (95.7) | 110 (94.8) | 334 (95.2) |
| Tender joint count (68 joints) | 24.3 ± 15.6 | 24.0 ± 14.6 | 21.5 ± 14.0 | 23.3 ± 14.8 |
| Swollen joint count (68 joints) | 16.7 ± 12.6 | 17.1 ± 13.1 | 15.6 ± 11.8 | 16.5 ± 12.5 |
| CRP - mg/dL | 18.12 ± 28.59 | 15.39 ± 18.59 | 16.84 ± 20.73 | 16.80 ± 23.07 |
| Patient global assessment | 62.0 ± 20.9 | 56.3 ± 22.6 | 58.4 ± 19.4 | 59.0 ± 21.0 |
| (0-100 mm VAS) | | | | |
| Physician global assessment | 58.0 ± 19.2 | 58.9 ± 21.1 | 59.1 ± 19.9 | 58.7 ± 20.0 |
| (0-100 mm VAS) | | | | |
| DAS28 (CRP) | 5.53 ± 1.25 | 5.46 ± 1.15 | 5.32 ± 1.33 | 5.44 ± 1.25 |
| HAQ-DI | 1.45 ± 0.68 | 1.42 ± 0.66 | 1.38 ± 0.65 | 1.42 ± 0.66 |
| Pain (0-100 mm VAS) | 61.6 ± 21.4 | 60.4 ± 21.5 | 58.6 ± 18.4 | 60.2 ± 20.4 |
| Physical function (0-100, | 38.5 ± 25.94 | 41.6 ± 25.64 | 39.1 ± 24.49 | 39.7 ± 25.3 |
| Short Form-36 subscale) | | | | |

Plus-minus values are means ± SD.
CRP = C-reactive protein,
DAS = Disease Activity Score,
HAQ-DI = Health Assessment Questionnaire-Disability Index,
MTX = methotrexate,
RA = rheumatoid arthritis,
RF = rheumatoid factor,
ROW = rest of the world,
SD = standard deviation,
VAS = visual analog scale

The number of patients entering the withdrawal period was 84/119 (70.6%), 66/116 (56.9%) and 73/116 (62.9%) in the abatacept plus MTX, abatacept monotherapy and MTX arms, respectively.

### Signs and Symptoms

### Abatacept Plus MTX Versus MTX During Treatment Period

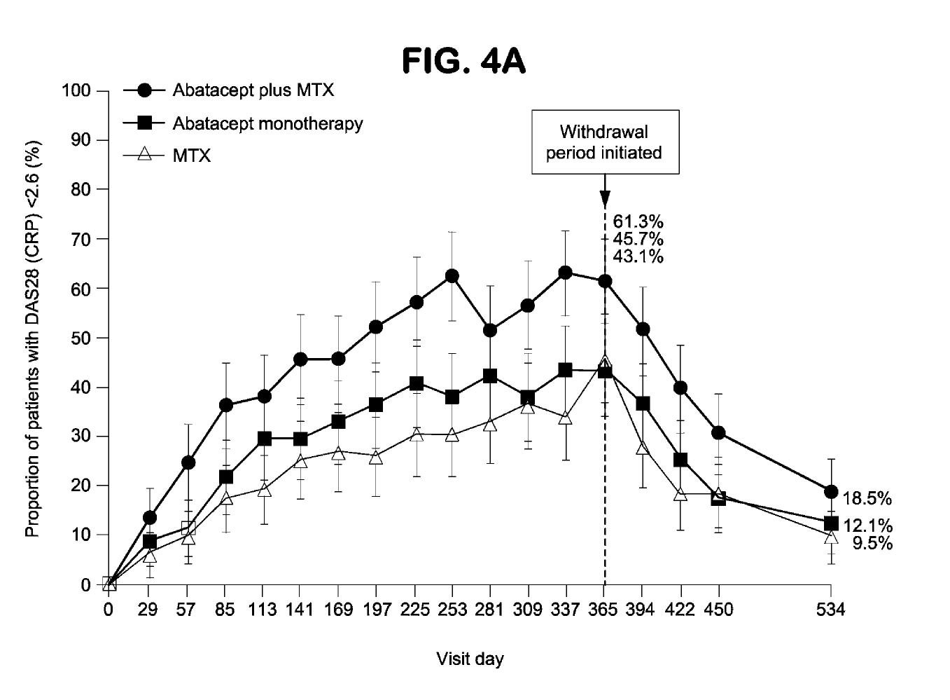

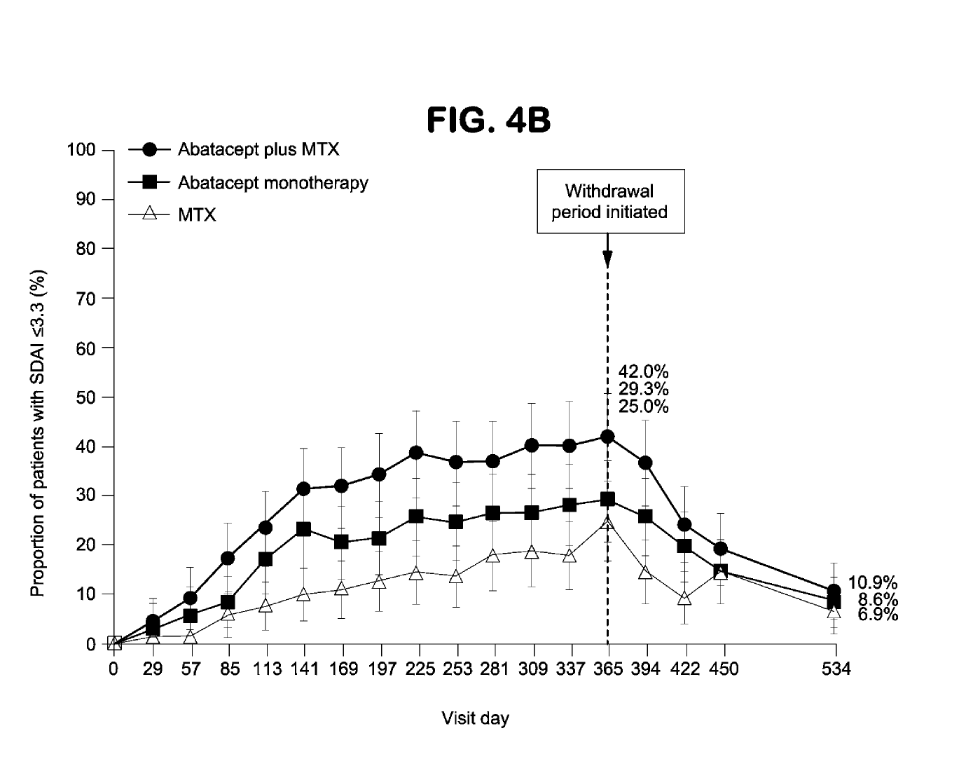

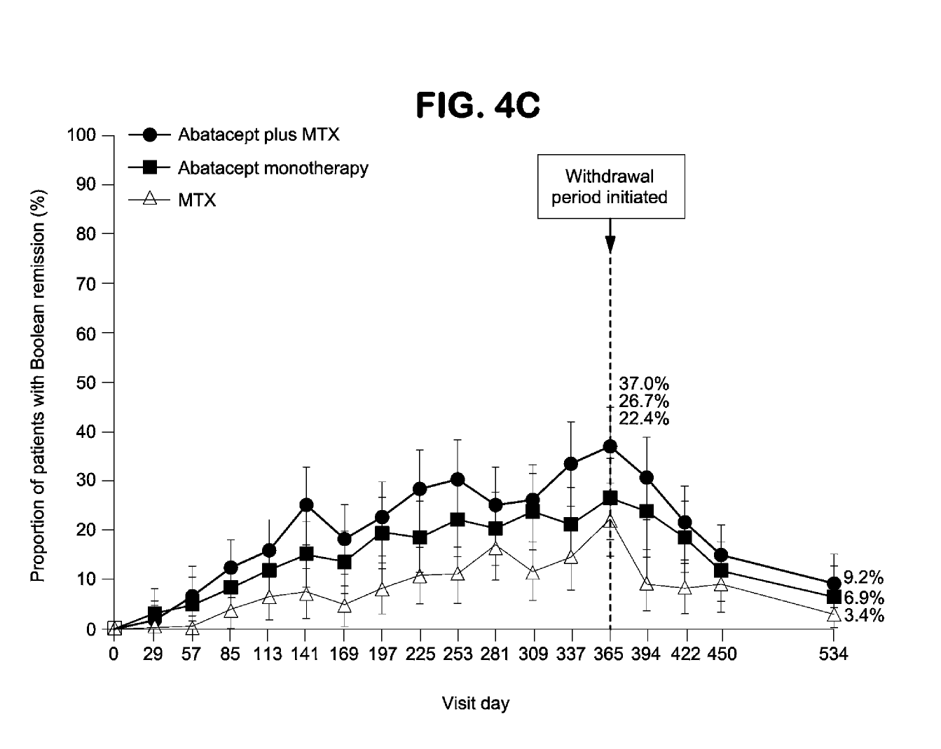

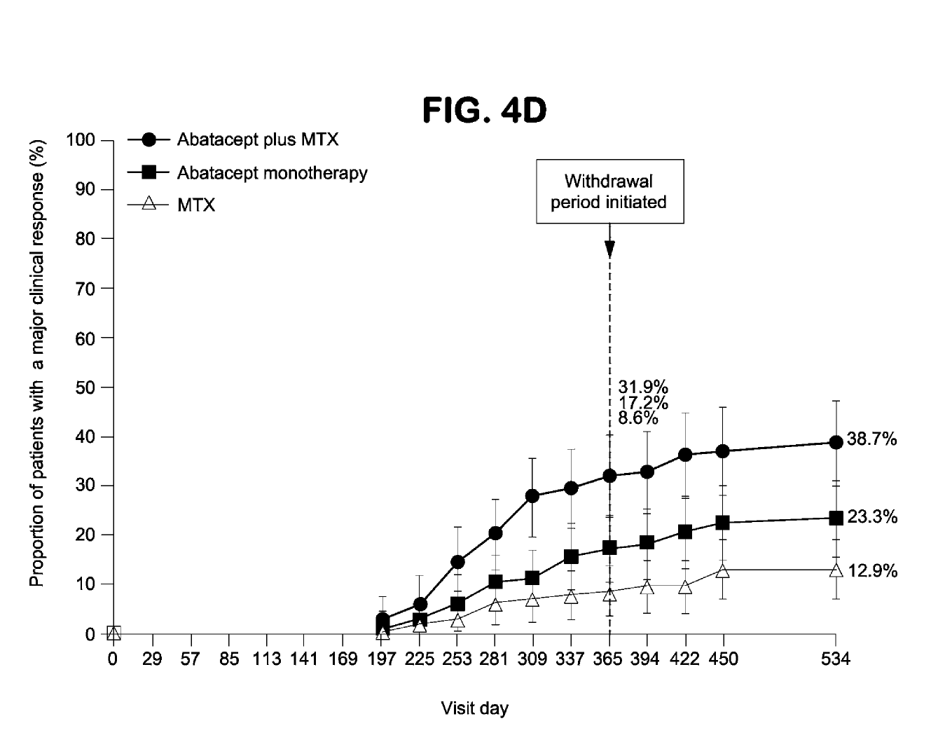

Abatacept plus MTX achieved statistically significantly higher rates of DAS-defined remission versus MTX at Month 12 (70/115 [60.9%] patients vs 52/115 [45.2%] patients; odds ratio [OR; 95% CI]: 2.01 [1.18, 3.43], P=0.010). Numerically higher DAS-defined remission rates were observed in the abatacept plus MTX group versus MTX from Day 57, which were maintained over time for the rest of the treatment period (see FIG. 4a). A post hoc analysis of the overall treatment effect over the 12 months of the treatment period in change from baseline in DAS28 (CRP) demonstrated an estimated treatment difference (95% CI) of −0.52 (−0.74, −0.30) for abatacept plus MTX versus MTX.

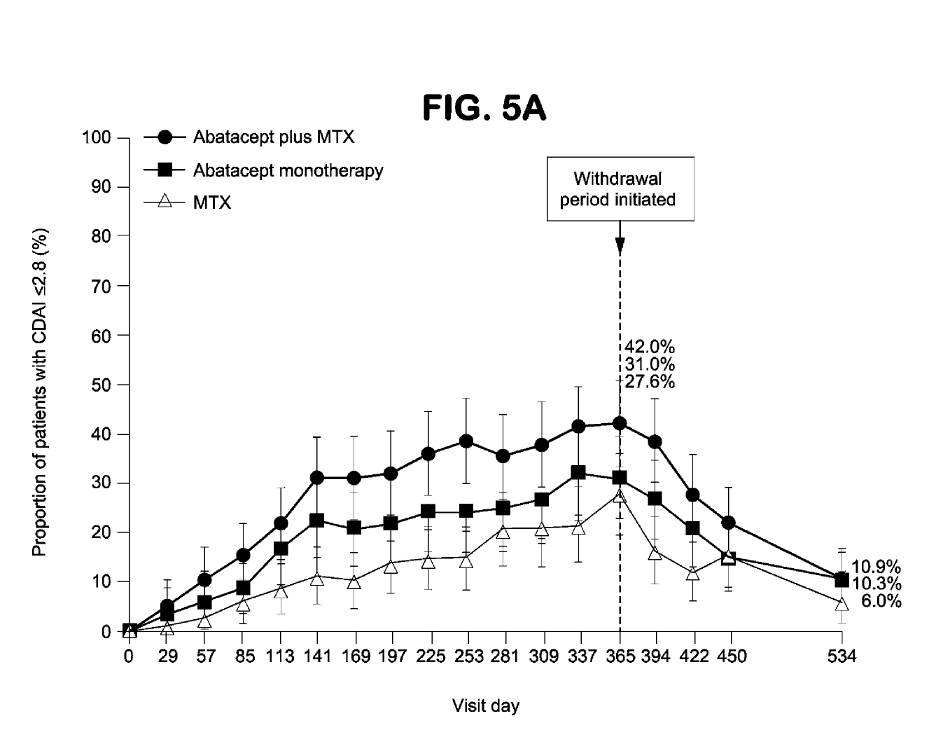

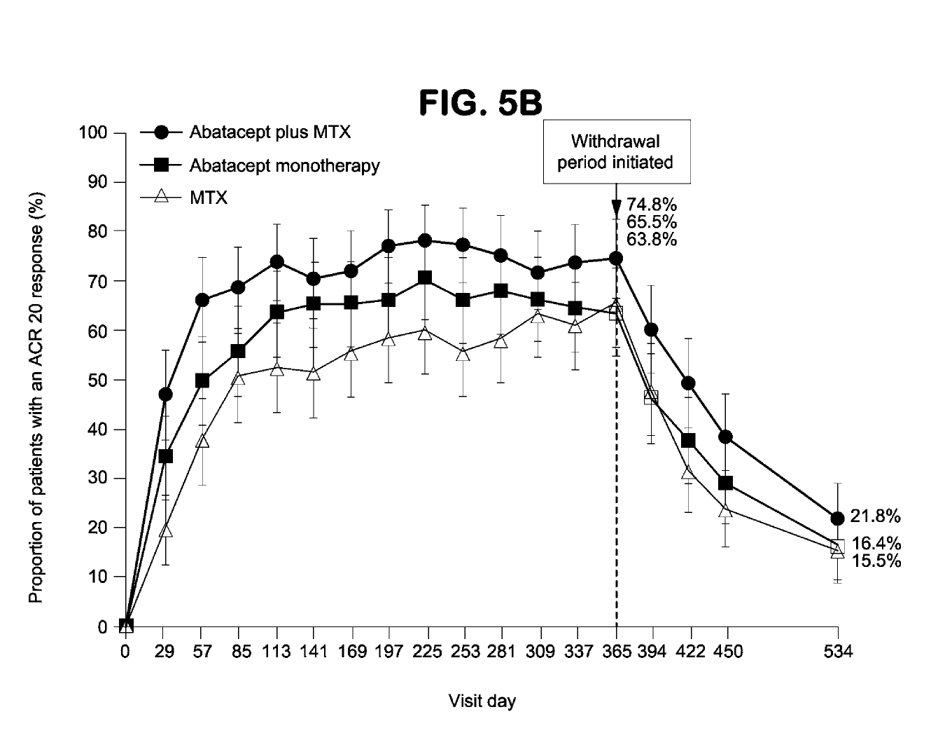

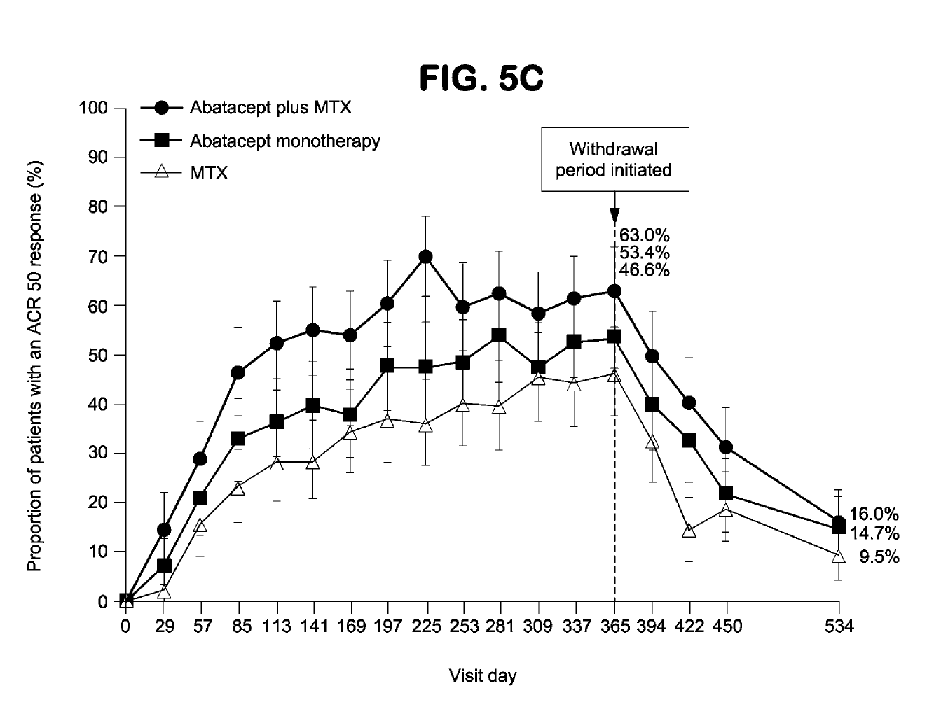

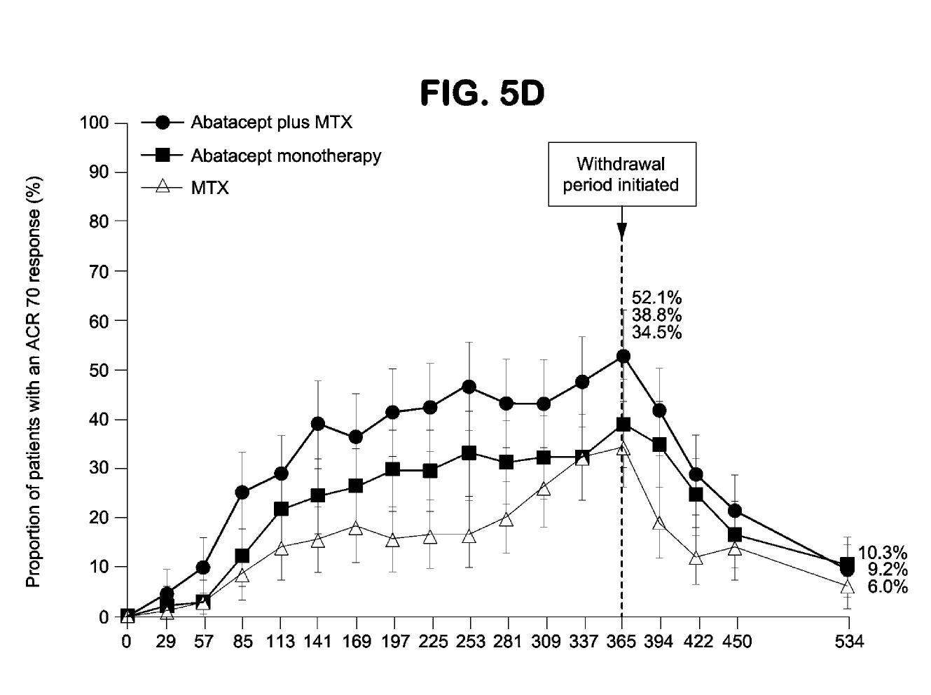

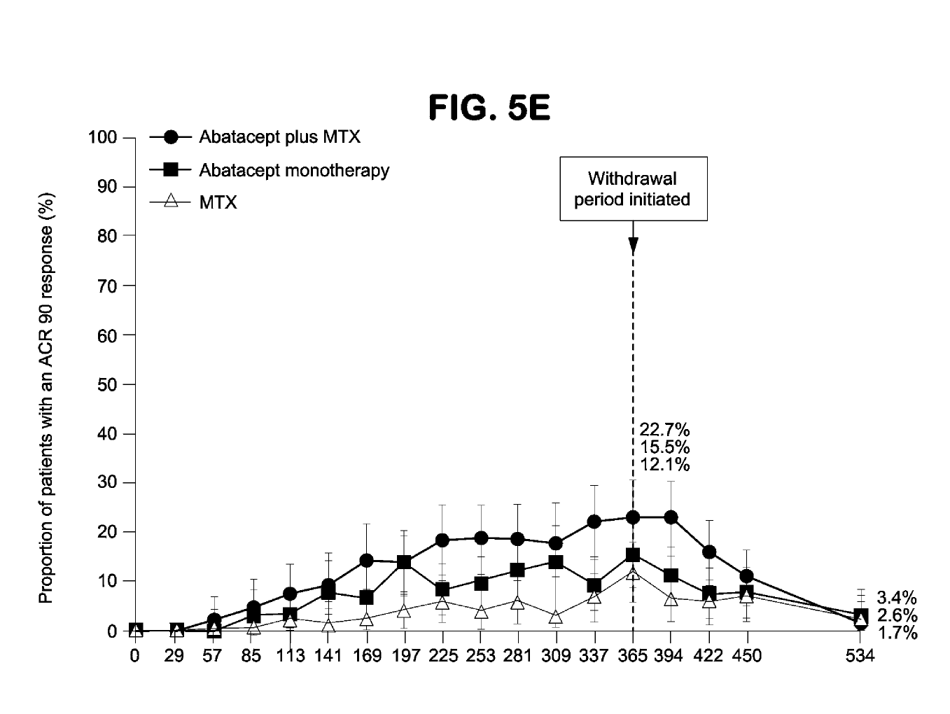

The proportion of patients achieving other remission endpoints (including Simplified Disease Activity Index [SDAI], Clinical Disease Activity Index [CDAI], and Boolean remission), American College of Rheumatology (ACR) responses and major clinical response (MCR) were numerically greater for abatacept plus MTX versus MTX over time (see FIG. 4A-D and FIG. 5A-E). As shown in Table 10 below, HAQ-DI response rates at Month 12 were 65.5% versus 44.0%, respectively.

TABLE 10
Proportion of Patients with Response on
Health Assessment Questionnaire-Disability
Index (HAQ-DI) at Months 12 and 18.*

| | HAQ-DI Response (≧0.3) | |
|---|---|---|
| | Month 12 | Month 18 |
| Abatacept plus MTX | 78 (65.5) (57.0, 74.1) | 26 (21.8) (14.4, 29.3) |
| Abatacept monotherapy | 61 (52.6) (43.5, 61.7) | 19 (16.4) (9.6, 23.1) |
| MTX | 51 (44.0) (34.9, 53.0) | 12 (10.3) (4.8, 15.9) |

*Values are no. (%) (95% CI).
CI = confidence interval;
MTX = methotrexate.

### Abatacept Monotherapy Versus MTX During Treatment Period

Abatacept monotherapy resulted in a similar proportion of patients achieving DAS-defined remission at Month 12 compared with MTX (48/113 [42.5%] vs. 52/115 [45.2%]). However, over time, DAS-defined remission rates were numerically higher for abatacept monotherapy (see FIG. 4A) at most other time points. In fact, as determined by post hoc analysis, the overall estimated treatment difference (95% CI) between abatacept monotherapy versus MTX in change from baseline in DAS28 (CRP) was −0.26 (−0.11, −0.48). Additionally, abatacept monotherapy demonstrated numerically higher rates of CDAI, SDAI, Boolean remission and ACR20/50/70 and MCR rates versus MTX over time (FIG. 4A-D and FIG. 5A-E) and HAQ-DI (Table 10 above).

### Abatacept Plus MTX and Abatacept Monotherapy Versus MTX During Withdrawal Period

Abatacept plus MTX achieved statistically significantly higher rates of DAS-defined remission versus MTX at both Months 12 and 18 (17/115 [14.8%] patients vs 9/115 [7.8%] patients; OR [95% CI]: 2.51 [1.02, 6.18], P=0.045). The proportion of patients achieving DAS-defined remission at both Months 12 and 18 was 14/113 (12.4%) vs 9/115 (7.8%) for abatacept monotherapy and MTX groups, respectively (analysis included only patients with DAS28 (CRP) available at baseline).

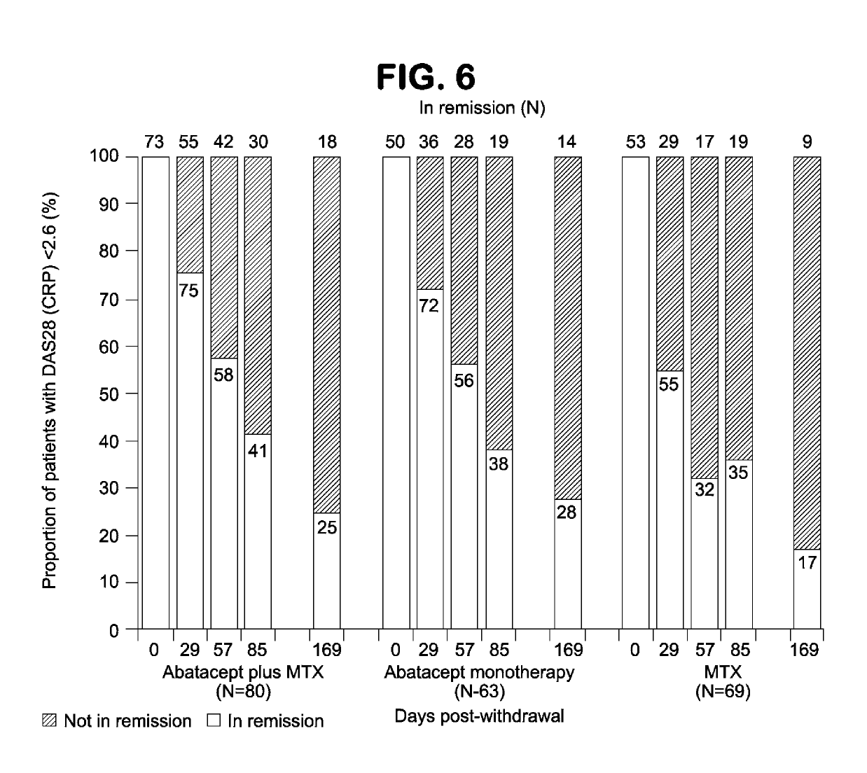

Of the patients who entered the withdrawal period, 73, 50 and 53 patients in each treatment group were in DAS-defined remission at Month 12. Of these, 18/73 (24.7%), 14/50 (28%) and 9/53 (16.9%) remained in DAS-defined remission at Month 18 (see FIG. 6).

A post hoc analysis shown in Table 11 below, indicated that, in both abatacept treatment arms, the proportions of patients with sustained DAS-defined remission following treatment withdrawal were numerically higher in patients who had lower baseline DAS28 (CRP), HAQ-DI and shorter symptom duration; this was not the case in the MTX arm.

TABLE 11
Proportion of Patients with DAS-defined Remission
(DAS28 [CRP] < 2.6) at Both Months 12 and
18 by Baseline Characteristic Subgroup (Post Hoc Analyses)

| | Abatacept | Abatacept | |
|---|---|---|---|
| | plus MTX | Monotherapy | MTX |
| Baseline characteristic | (N = 119) | (N = 116) | (N = 116) |
| DAS28 (CRP) | | | |
| Missing - no./N (%) | 1/4 (25.0) | 0/3 (0) | 0/1 (0) |
| ≦Median (5.4) - no./N (%) | 14/56 (25.0) | 12/56 (21.4) | 6/60 (10.0) |
| >Median (5.4) - no./N (%) | 3/59 (5.1) | 2/57 (3.5) | 3/55 (5.5) |
| HAQ-DI, n (%) | | | |
| Missing - no./N (%) | 3/6 (50.0) | 1/3 (33.3) | 0/11 (0) |
| ≦Median (1.375) - no./N (%) | 12/58 (20.7) | 10/59 (16.9) | 4/56 (7.1) |
| >Median (1.375) - no./N (%) | 3/55 (5.5) | 3/54 (5.6) | 5/49 (10.2) |
| Symptom duration | | | |
| ≦Median (0.37 years) - | 12/58 (20.7) | 7/50 (14.0) | 5/69 (7.2) |
| no./N (%) | | | |
| >Median (0.37 years) - | 6/61 (9.8) | 7/66 (10.6) | 4/47 (8.5) |
| no./N (%) | | | |
| ≦6 Months - no./N (%) | 14/70 (20.0) | 11/71 (15.5) | 7/77 (9.1) |
| >6 Months - no./N (%) | 4/49 (8.2) | 3/45 (6.7) | 2/39 (5.1) |
| Pain (100 mm VAS) | | | |
| Missing, no./N (%) | 3/6 (50.0) | 1/3 (33.3) | 0/11 (0.0) |
| ≦Median (62), no./N (%) | 11/48 (22.9) | 8/58 (13.8) | 7/60 (11.7) |
| >Median (62), no./N (%) | 4/65 (6.2) | 5/55 (9.1) | 2/45 (4.4) |
| Erosion | | | |
| Missing - no./N (%) | 1/15 (6.7) | 0/14 (0) | 2/13 (15.4) |
| ≦Median (4.5) - no./N (%) | 7/50 (14.0) | 10/58 (17.2) | 4/53 (7.5) |
| >Median (4.5) - no./N (%) | 10/54 (18.5) | 4/44 (9.1) | 3/50 (6.0) |
| ≦Q1 (1.5) - no./N (%) | 4/23 (17.4) | 5/28 (17.9) | 2/30 (6.7) |
| >Q1 (1.5)-Q2 (4.5) - | 3/27 (11.1) | 5/30 (16.7) | 2/23 (8.7) |
| no./N (%) | | | |
| >Q2 (4.5)-Q3 (8.5) - | 8/25 (32.0) | 3/23 (13.0) | 2/23 (8.7) |
| no./N (%) | | | |
| >Q3 (8.5) - no./N (%) | 2/29 (6.9) | 1/21 (4.8) | 1/27 (3.7) |
| Osteitis | | | |
| Missing - no./N (%) | 1/15 (6.7) | 0/14 (0) | 2/13 (15.4) |
| ≦Median (0.5) - no./N (%) | 8/54 (14.8) | 10/47 (21.3) | 4/54 (7.4) |
| >Median (0.5) - no./N (%) | 9/50 (18.0) | 4/55 (7.3) | 3/49 (6.1) |
| ≦Q1 (0) - no./N (%) | 6/41 (14.6) | 9/43 (20.9) | 4/49 (8.2) |
| >Q1 (0)-Q2 (0.5) - no./N (%) | 2/13 (15.4) | 1/4 (25.0) | 0/5 (0) |
| >Q2 (0.5)-Q3 (5) - no./N (%) | 7/25 (28.0) | 2/27 (7.4) | 2/26 (7.7) |
| >Q3 (5) - no./N (%) | 2/25 (8.0) | 2/28 (7.1) | 1/23 (4.3) |
| Synovitis | | | |
| Missing - no./N (%) | 1/15 (6.7) | 0/14 (0) | 2/13 (15.4) |
| ≦Median (4.5) - no./N (%) | 12/57 (21.1) | 9/52 (17.3) | 4/47 (8.5) |
| >Median (4.5) - no./N (%) | 5/47 (10.6) | 5/50 (10.0) | 3/56 (5.4) |
| ≦Q1 (2) - n/N (%) | 5/30 (16.7) | 4/24 (16.7) | 3/24 (12.5) |
| >Q1 (2)-Q2 (4.5) - no./N (%) | 7/27 (25.9) | 5/28 (17.9) | 1/23 (4.3) |
| >Q2 (4.5)-Q3 (8.5) - | 3/23 (13.0) | 4/33 (12.1) | 1/30 (3.3) |
| no./N (%) | | | |
| >Q3 (8.5) -, no./N (%) | 2/24 (8.3) | 1/17 (5.9) | 2/26 (7.7) |

CRP = C-reactive protein,
DAS = Disease Activity Score,
HAQ-DI = Health Assessment Questionnaire-Disability Index,
MTX = methotrexate,
Q = quartile,
VAS = visual analogue scale

Similarly, as shown in Table 12 below, the baseline factors associated with DAS-defined remission at Months 12 and 18 above, were identified as baseline factors associated with DAS-defined remission at Month 12, also in the abatacept arm.

TABLE 12
Baseline Characteristics of Patients With or Without Drug-free DAS-defined
Remission (DAS28 [CRP] <2.6) at Month 18 Following Attainment of
Remission at Month 12 (Post Hoc Analyses)

| | DAS-defined Remission | | | | | |
|---|---|---|---|---|---|---|
| | Abatacept | | Abatacept | | | |
| | plus MTX | | Monotherapy | | MTX | |
| | At Month | At Both | At Month | At both | At Month | At Both |
| | 12 but not | Months | 12 but not | Months | 12 but not | Months |
| Parameter | Month18 | 12 and 18 | Month18 | 12 and 18 | Month18 | 12 and 18 |
| (Mean) | (N = 55) | (N = 18) | (N = 36) | (N = 14) | (N = 44) | (N = 9) |
| Symptom | 0.6 | 0.4 | 0.7 | 0.5 | 0.4 | 0.4 |
| duration at | | | | | | |
| baseline - yr | | | | | | |
| Tender joint | 14.5 | 9.1 | 15.6 | 8.3 | 12.8 | 13.4 |
| count (28 joints) | | | | | | |
| at baseline | | | | | | |
| Swollen joint | 12.0 | 6.7 | 14.1 | 6.4 | 10.6 | 9.2 |
| count (28 joints) | | | | | | |
| at baseline | | | | | | |
| Pain (0-100 mm | 62.8 | 51.9 | 59.5 | 50.5 | 59.8 | 50.7 |
| VAS) | | | | | | |
| HAQ-DI | 1.5 | 1.1 | 1.4 | 1.0 | 1.3 | 1.5 |
| CRP at baseline - | 16.8 | 11.2 | 13.9 | 7.2 | 13.5 | 24.9 |
| mg/dL | | | | | | |
| DAS28 (CRP) | 5.7 | 4.5 | 5.7 | 4.3 | 5.2 | 5.4 |
| MRI synovitis | 6.0 | 4.4 | 5.6 | 4.2 | 5.8 | 5.2 |
| MRI osteitis | 5.1 | 2.5 | 4.6 | 4.0 | 3.7 | 2.7 |
| MRI erosion | 6.2 | 5.0 | 5.7 | 3.4 | 6.3 | 4.7 |

CRP = C-reactive protein,
DAS = Disease Activity Score,
HAQ-DI = Health Assessment Questionnaire-Disability Index,
MRI = magnetic resonance imaging,
MTX = methotrexate,
VAS = visual analog scale

Patients receiving abatacept also had more time with DAS28 (CRP)<2.6 than patients receiving MTX during the treatment period (10.2 vs 8.1 months for abatacept plus MTX; 8.9 vs 6.6 months for abatacept monotherapy; 5.8 vs 5.7 months for MTX).

### Effect on Structural Damage

Radiographic changes measured by MRI in each of the treatment groups were consistent with clinical efficacy outcomes. Abatacept plus MTX and abatacept monotherapy resulted in numerically greater decreases from baseline in synovitis and osteitis scores, and abatacept plus MTX resulted in less progression of erosion score, than MTX at 12 months (see FIG. 7A-C).

### DISCUSSION

In AVERT, patients had highly active disease and poor prognostic markers; 95% of patients were anti-CCP-2 positive and rheumatoid factor positive, a combination associated with enhanced probability of joint damage and disease progression (Goronzy, J. J. et al., “Prognostic markers of radiographic progression in early rheumatoid arthritis”, Arthritis Rheum., 50:43-54 (2004); Kroot, E. J. et al., “The prognostic value of anti-cyclic citrullinated peptide antibody in patients with recent-onset rheumatoid arthritis”, Arthritis Rheum., 43:1831-1835 (2000)). Abatacept plus MTX achieved robust efficacy versus MTX, as demonstrated by multiple measures of remission and HAQ-DI, and consistent structural benefits. While joint counts can be subjective and month-by-month variability was evident, the MRI results provide an objective measure of comparative efficacy in support of the clinical endpoints.

AVERT provides a large dataset assessing abatacept monotherapy, which is of interest because many patients cannot tolerate MTX: approximately 30% of patients receive biologics as monotherapy (Emery, P. et al., “Biologic and oral disease-modifying antirheumatic drug monotherapy in rheumatoid arthritis”, Ann. Rheum. Dis., 72:1897-1904 (2013)). A similar number of patients receiving abatacept achieved DAS-defined remission versus MTX at Month 12, but the overall data showed that abatacept monotherapy had numerically higher benefit compared with MTX. The MRI findings for abatacept monotherapy also demonstrated a numerically greater benefit on osteitis and synovitis compared with MTX alone at Month 12.

Following withdrawal of all therapy, a small but significant number of patients sustained drug-free remission following prior treatment with abatacept plus MTX compared with MTX alone. The data indicate that, with abatacept plus MTX treatment, one in four patients was able to maintain drug-free remission through 6 months. This effect is not a consequence of the half-life of abatacept (14.3 days) as assessments were performed up to 6 months after the withdrawal of all treatment (>5 half-lives). Moreover, the post hoc analyses of the patients that sustained drug-free remission suggest that patients with shorter symptom duration and lower disease activity at baseline, or longer sustained DAS-defined remission prior to treatment withdrawal, were more likely to maintain drug-free remission. These associations were observed specifically in both abatacept arms, suggesting a biologic effect was responsible.

The DAS-defined remission cut-off of <2.6, although corresponding to the American Rheumatology Association definition of clinical remission in RA (Fransen, J. et al., “Remission in rheumatoid arthritis: agreement of the disease activity score (DAS28) with the ARA preliminary remission criteria”, Rheumatology (Oxford), 43:1252-1255 (2004)), has now been replaced with other measures of remission (Felson, D. T. et al., “American College of Rheumatology/European League against Rheumatism provisional definition of remission in rheumatoid arthritis for clinical trials”, Ann. Rheum. Dis., 70:404-413 (2011)); such as SDAI and Boolean which are also reported here. The cut-off is based on erythrocyte sedimentation rate (ESR), and a CRP cut-off has yet to be defined.

In AVERT, CRP was interchanged with ESR to reduce the variability of the acute phase reactant and aid standardization across study centers. Data are obtained from patients with early RA with active disease and poor prognostic factors, which limit its generalizability to the overall RA population. The withdrawal analyses were limited by the small number of patients who remained in the withdrawal period. The gradual tapering of RA medication may result in higher remission rates than the rapid withdrawal of all RA therapy applied in AVERT and will be assessed in other trials.

In conclusion, AVERT establishes the benefit of abatacept treatment in combination with MTX in an early RA population and suggests that, in early RA, drug-free remission may be possible following treatment with abatacept. The novel achievement of sustained remission following withdrawal of all RA therapy is suggestive of an underlying effect of abatacept's mechanism on autoimmune processes. A withdrawal treatment strategy is a highly desirable goal for patients and physicians in the long-term treatment of RA. Treat-to-remission is now a well accepted goal of RA therapy.

What is claimed is:

1. A method for achieving drug free remission in subjects with early rheumatoid arthritis (RA) comprising
i) administering to the subject an effective amount of CTLA4 molecule or pharmaceutical composition thereof until Disease Activity Score Calculator for Rheumatoid Arthritis (DAS)-defined remission is achieved, and
ii) withdrawing all RA therapy,
wherein the CTLA4 molecule binds CD80 and/or CD86 and comprises an extracellular domain of CTLA4 as shown in SEQ ID NO:2 beginning with alanine at position 26 or methionine at position 27 and ending with aspartic acid at position 150.

2. The method of claim 1 further comprising an amino acid sequence which alters the solubility or affinity of the CTLA4 molecule.

3. The method of claim 2, wherein the amino acid sequence which alters the solubility or affinity comprises an immunoglobulin.

4. The method of claim 3, wherein the immunoglobulin is an immunoglobulin constant region or portion thereof.

5. The method of claim 4, wherein the immunoglobulin constant region or portion thereof is mutated to reduce effector function.

6. The method of claim 4, wherein the immunoglobulin constant region or portion thereof comprises a hinge, CH2 and CH3 regions of a human or monkey immunoglobulin molecule.

7. The method of claim 5, wherein the immunoglobulin constant region or portion thereof comprises a hinge, CH2 and CH3 regions of a human or monkey immunoglobulin molecule.

8. A method for achieving drug free remission in subjects with early rheumatoid arthritis comprising
i) administering to the subject an effective amount of CTLA4 molecule or pharmaceutical composition thereof, comprising an amino acid sequence beginning with methionine at position 27 or alanine at position 26 and ending with lysine at position 383 of SEQ ID NO:2 until DAS-defined remission is achieved, and
ii) withdrawing all RA therapy.

9. The method of claim 1 or 8, wherein DAS-defined remission is characterized as DAS28 (C-reactive protein[CRP]) less than 2.6 after 12 months of treatment.

10. The method of claim 1 or 8, wherein the CTLA4 pharmaceutical composition is a subcutaneous formulation administered at 125 mg/week subcutaneously.

11. The method of claim 1 or 8, wherein early RA is characterized as having active clinical synovitis of ≧2 joints for ≧8 weeks, DAS28 (CRP) ≧3.2 and anti-citrullinated peptide (CCP)-2 antibody positivity.

12. A method of identifying subjects with early RA who are likely to achieve drug-free remission after treatment with CTLA4 molecules or pharmaceutical compositions thereof comprising selecting subjects with active clinical synovitis of ≧2 joints for ≧8 weeks, DAS28 (CRP) ≧3.2 and anti-citrullinated peptide (CCP)-2 antibody positivity.

13. A method of inhibiting structural damage in subjects with early RA comprising
i) administering to the subject an effective amount of CTLA4 molecule or pharmaceutical composition thereof until DAS-defined remission is achieved, and
ii) withdrawing all RA therapy,
wherein the CTLA4 molecule binds CD80 and/or CD86 and comprises an extracellular domain of CTLA4 as shown in SEQ ID NO:2 beginning with alanine at position 26 or methionine at position 27 and ending with aspartic acid at position 150.

14. The method of claim 13 wherein structural damage is assessed by erosion, osteitis and/or synovitis scoring of the wrist and hand.
